(19) United States
(12) Patent Application Publication
(10) Pub. No.: US 2026/0124318 A1
(43) Pub. Date: May 7, 2026

(54) POLYMERIC MATERIAL AND PREPARATION METHOD THEREOF, AND DRUG-LOADING MATERIAL

(71) Applicant: Southern University of Science and Technology, Shenzhen (CN)

(72) Inventors: Qiongyu GUO, Shenzhen (CN); Yutao MA, Shenzhen (CN)

(73) Assignee: Southern University of Science and Technology, Shenzhen (CN)

(21) Appl. No.: 19/435,614

(22) Filed: Dec. 29, 2025

Related U.S. Application Data
(63) Continuation-in-part of application No. PCT/CN2024/111441, filed on Aug. 12, 2024.

(30) Foreign Application Priority Data
Jun. 29, 2023 (CN) 202310790628.5

(51) Int. Cl. A61K 47/69; A61K 31/704; A61K 31/78; A61K 47/61

(52) U.S. Cl. CPC A61K 47/6927; A61K 31/704; A61K 31/78; A61K 47/61

(57) ABSTRACT

A polymeric material, a preparation method thereof, and a drug-loading material are provided. The polymeric material includes: a crosslinked polymer; and multiple polyelectrolytes, which are grafted onto the three-dimensional network structure of the crosslinked polymer through chemical bonds, each of the polyelectrolytes containing multiple ionizable groups, so that the multiple ionizable groups form local potentials upon ionization to induce aggregation of particles with opposite charges. Compared with conventional drug-loaded microsphere embolic agents, the polymeric material provided by the present disclosure can increase its adsorption amount and adsorption efficiency for particles with opposite charges. When the particles with opposite charges are drug particles, using the polymeric material provided by the present disclosure can increase the drug-loading capacity and shorten the drug-loading time.

### RELATED APPLICATIONS

This application is a Continuation-in-Part (CIP) application of PCT application No. PCT/CN2024/111441, filed on August 12, 2024, which claims the benefit of priority to Chinese Patent Application No. 202310790628.5, filed on June 29, 2023, and the content of which is incorporated herein by reference in entirety.

### TECHNICAL FIELD

The present disclosure relates to the technical field of medical polymer materials, and particularly relates to a polymeric material and a preparation method thereof, and a drug-loading material.

### BACKGROUND

Liver cancer is a common cancer worldwide, and transcatheter arterial chemoembolization (TACE) is the preferred strategy for treating middle and late-stage liver cancer. TACE is a technique in which, under the guidance of medical imaging equipment, a drug-loaded embolic agent is injected via a catheter to a target location; the embolic agent blocks the blood supply, while the drug is released from the embolic agent to achieve the intended therapeutic purpose. It has the advantages of being minimally invasive, accurately positioned, and having few side effects. Embolization therapy has achieved good results in treating malignant tumors, uterine fibroids, hemangiomas, vascular malformations, and hemostasis. In recent years, an increasing number of embolic materials have been approved by the China Food and Drug Administration (CFDA) for clinical embolization of blood vessels, tumors, and other indications, including polyvinyl alcohol particulate embolic agents, polyvinyl alcohol drug-loading embolic microspheres, and medical polyether polyurethane embolic agents. TACE technology can achieve chemotherapy effects while performing embolization treatment. To achieve better embolization-chemotherapy effects, higher requirements are placed on the material properties of the embolic agents.

The principle for adsorbing drugs in the current preparation of drug-loading microsphere embolic agents is mainly as follows: by using electrostatic adsorption, acidic groups are modified onto the original materials; after ionization, the acidic groups (such as carboxyl groups, etc.) can carry negative charges, enabling the material to have functions such as drug loading. Clinically used embolic agents include polyvinyl alcohol polymer hydrogel microspheres modified with sulfonic acid groups (DC Bead), polyvinyl alcohol embolic microspheres (Callisphere), and embolic microspheres (Hepasphere), etc.

At present, in the modified materials used for drug-loading microspheres, the negatively charged groups within the molecules are distributed irregularly in space. Although the number of negative groups is large, their density is not high, and it takes 30 min to 2 h or even more than 2 h to achieve complete drug loading, resulting in excessively long loading times. Moreover, degradable microspheres that have received increasing attention in recent years are limited by the structures of degradable materials. Due to steric hindrance and other factors, the total amount of negatively charged groups within the modified molecules is limited and irregularly distributed, leading to low drug-loading capacity and the inability to ensure sufficient loading of anticancer drugs for tumor treatment. FIG. 1 shows a schematic diagram of the microstructure of an existing drug-loading microsphere. Using the drug-loading microsphere shown in FIG. 1 as an example, carboxyl groups are grafted onto the framework of the polymer material and are dispersed throughout, presenting an irregular distribution. Even though the framework of the polymer material contains many active sites for grafting carboxyl groups, the density of carboxyl groups on the framework of the polymer material is still relatively low.

### SUMMARY

To solve the problems in the existing technology described above, the main purpose of the present disclosure is to provide a polymeric material and a preparation method thereof, and a drug-loading material.

To achieve the above purpose, in a first aspect, the present disclosure provides a polymeric material, including: a crosslinked polymer; and a plurality of polyelectrolytes grafted via chemical bonds onto a three-dimensional network structure of the crosslinked polymer, where each of the polyelectrolytes includes a plurality of ionizable groups, and the ionizable groups form a local potential after ionization to induce aggregation of particles having an opposite electric charge.

In a second aspect, the present disclosure provides a method for preparing a polymeric material, including: mixing a crosslinking precursor, a crosslinking agent, and polyelectrolytes; and performing a crosslinking polymerization reaction under an external condition stimulation to form the polymeric material, where polymeric material includes: a crosslinked polymer, and a plurality of the polyelectrolytes grafted via chemical bonds onto a three-dimensional network structure of the crosslinked polymer, where each of the polyelectrolytes includes a plurality of ionizable groups, so that the ionizable groups form a local potential after ionization to induce aggregation of particles having an opposite electric charge.

In a third aspect, the present disclosure provides a drug-loading material, including: a polymeric material, including: a crosslinked polymer, and a plurality of polyions grafted by chemical bonds onto a three-dimensional network structure of the crosslinked polymer; and a plurality of drug particles, adsorbed onto the polymeric material through electrostatic interaction, where each of the polyions includes a plurality of charged groups, and the plurality of charged groups forms a local potential to induce aggregation of the drug particles having opposite electric charges.

In the technical solutions of the present disclosure, a polyelectrolyte(s) is grafted onto the three-dimensional network structure of a crosslinked polymer, which can introduce more ionizable groups into the three-dimensional network structure of the crosslinked polymer, increase the total number of ionizable groups carried by the polymeric material, as well as the local density of the ionizable groups, and use the counterion condensation effect to form a local potential, thereby increasing the adsorption amount and adsorption efficiency of the polymer material for particles carrying an opposite charge. When the particles carrying an opposite charge are drug particles, the polymeric material provided by the present disclosure can increase the drug-loading capacity and shorten the drug-loading time.

### BRIEF DESCRIPTION OF THE DRAWINGS

In order to more clearly illustrate the technical solutions in the embodiments of the present disclosure, the drawings needed for the description of the embodiments will be briefly introduced below. Obviously, the drawings in the following description are only some exemplary embodiments of the present disclosure. For a person of ordinary skill in the art, other drawings can also be obtained based on these drawings without creative effort.

FIG. 1 shows a schematic diagram of the microscopic structure of an existing drug-loaded microsphere;

FIG. 2 shows a schematic diagram of the microscopic structure of a polymeric material provided according to the present disclosure;

FIG. 3 shows a schematic diagram of the microscopic structure of a drug-loading material provided according to the present disclosure;

FIG. 4 shows a microscopic photograph of a polymeric material prepared according to some exemplary embodiments of the present disclosure;

FIG. 5 shows a microscopic photograph of a polymeric material prepared according to some exemplary embodiments of the present disclosure;

FIG. 6 shows a schematic diagram of the electronegativity corresponding to different polyacrylic acid contents of a polymeric material prepared according to some exemplary embodiments of the present disclosure;

FIG. 7 shows a microscopic photograph of a polymeric material prepared according to some exemplary embodiments of the present disclosure;

FIG. 8 shows a schematic diagram of the maximum drug loading corresponding to different polyacrylic acid contents of a polymeric material prepared according to some exemplary embodiments of the present disclosure;

FIG. 9 shows a schematic diagram of the drug-loading efficiency corresponding to different polyacrylic acid contents of a polymeric material prepared according to some exemplary embodiments of the present disclosure;

FIG. 10 shows a schematic diagram of the variation over time of the drug-loading efficiency corresponding to a polymeric material containing a specific polyacrylic acid content prepared according to some exemplary embodiments of the present disclosure;

FIG. 11 shows a schematic diagram of the drug-loading rate of a polymeric material prepared according to some exemplary embodiments of the present disclosure when containing different polyanion molecular weights;

FIG. 12 shows a schematic diagram of the degradation rate and drug release rate of a drug-loading material prepared according to some exemplary embodiments of the present disclosure under different in-vitro conditions;

FIG. 13 shows the drug release of a drug-loading material prepared according to some exemplary embodiments of the present disclosure in rabbit ear vascular embolization chemotherapy;

FIG. 14A shows the fluorescence signal on day 14 in rabbit ear vascular embolization chemotherapy corresponding to a drug-loading material prepared according to some exemplary embodiments of the present disclosure;

FIG. 14B shows the fluorescence signal on day 28 in rabbit ear vascular embolization chemotherapy corresponding to a drug-loading material prepared according to some exemplary embodiments of the present disclosure;

FIG. 15A shows the H&E-stained tissue section on day 14 in rabbit ear vascular embolization chemotherapy corresponding to a drug-loading material prepared according to some exemplary embodiments of the present disclosure;

FIG. 15B shows the H&E-stained tissue section on day 28 in rabbit ear vascular embolization chemotherapy corresponding to a drug-loading material prepared according to some exemplary embodiments of the present disclosure;

FIG. 15C shows the H&E-stained tissue section on day 90 in rabbit ear vascular embolization chemotherapy corresponding to a drug-loading material prepared according to some exemplary embodiments of the present disclosure;

FIG. 16A shows a microscopic photograph of a polymeric material according to some exemplary embodiments of the present disclosure when not loaded with a drug;

FIG. 16B shows a microscopic photograph of doxorubicin hydrochloride and the polymeric material just mixed according to some exemplary embodiments of the present disclosure;

FIG. 16C shows a microscopic photograph of a drug-loading material obtained after the polymeric material completes drug loading according to some exemplary embodiments of the present disclosure;

FIG. 17A shows a schematic diagram of the arrangement of a drug-loading material in a blood vessel according to some exemplary embodiments of the present disclosure;

FIG. 17B shows a schematic diagram of the arrangement of a drug-loading material in a blood vessel according to some exemplary embodiments of the present disclosure; and

FIG. 17C shows a schematic diagram of the arrangement of a drug-loading material in a blood vessel according to some exemplary embodiments of the present disclosure.

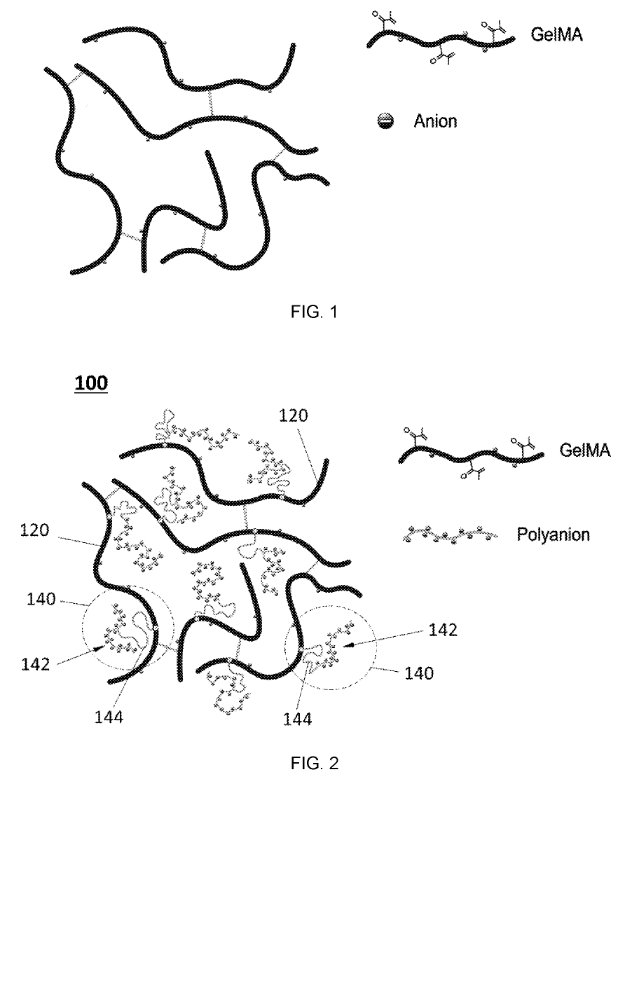

### DETAILED DESCRIPTION

For the purpose of facilitating the understanding of the present disclosure, the present disclosure will be described more comprehensively below with reference to the relevant drawings. The drawings illustrate some exemplary embodiments of the present disclosure. However, the present disclosure can be implemented in many different forms without departing from the core spirit of the present disclosure and is not limited to the embodiments described herein. On the contrary, the purpose of providing these embodiments is to make the disclosure of the present disclosure more thorough and comprehensive.

Unless otherwise defined, all technical and scientific terms used herein have the same meanings as those commonly understood by persons skilled in the art to which the present disclosure belongs. The terms used in the present disclosure are merely for the purpose of describing specific embodiments and are not intended to limit the present disclosure. The term “and/or” used herein includes any and all combinations of one or more of the listed items.

For the sake of convenience in description, the terms that may appear in the present disclosure are first explained as follows:

Crosslinked substance: also known as crosslinked polymer or crosslinked macromolecule, is a class of polymers having a three-dimensional network structure.

Crosslinking reaction: refers to a reaction in which two or more molecules (generally linear molecules) bond and crosslink with each other to form a more stable network-structured molecule (bulk molecule). This reaction transforms linear or lightly branched macromolecules into a three-dimensional network structure.

Polyelectrolyte (also known as polymer electrolyte) is a class of linear or branched synthetic or natural water-soluble macromolecules, whose structural units contain ionizable groups and exhibit good ionic conductivity. After dissolving, a polyelectrolyte can ionize into one polyion and many small ions with charges opposite to that of the polyion (also called counterions). For example, after dissolving, a polyelectrolyte can ionize into a negatively charged polyion (also called polyanion) and many cations with charges opposite to the polyanion; or a polyelectrolyte can ionize into a positively charged polyion (also called polycation) and many anions with charges opposite to the polycation. The electric property of the polyion obtained after polyelectrolyte ionization depends on the nature of the ionizable groups contained in the polyelectrolyte.

Electrostatic force refers to the long-range electric force generated by interactions between electrons. Electrostatic interactions include electrostatic attraction and electrostatic repulsion. Ionic bonds are formed through electrostatic interactions between cations and anions generated when atoms gain or lose electrons.

In a first aspect, the present disclosure provides a polymeric material, the polymeric material includes: a crosslinked polymer; and a plurality of polyelectrolytes, where the plurality of polyelectrolytes are grafted onto the three-dimensional network structure of the crosslinked polymer through chemical bonds, and each of the polyelectrolytes contains a plurality of ionizable groups, so that the plurality of ionizable groups form a local potential after ionization, inducing the aggregation of particles with opposite electric charges.

In the polymeric material provided by the present disclosure, the three-dimensional network structure of the crosslinked polymer is formed by the interweaving of a plurality of polymer chains, and multiple types of groups are also formed on each polymer chain. These groups can exhibit different chemical activities depending on their own chemical properties. The molecular chain of each polyelectrolyte contains a plurality of ionizable groups. The ionizable groups can be acidic groups, which, after ionization, can form polyanions (the polyanion contains a plurality of negatively charged groups) and a plurality of free cations (such as H⁺). In this case, the plurality of ionizable groups (acidic groups) on the polyelectrolyte can form a local low potential through the counterion condensation effect, thereby inducing the aggregation of particles with opposite electric charges (positively charged particles). The ionizable groups contained in the polyelectrolyte can also be basic groups, which, after ionization, can form polycations (the polycation contains a plurality of positively charged groups) and a plurality of free anions (such as OH⁻). In this case, the plurality of ionizable groups (basic groups) on the polyelectrolyte can form a local high potential through the counterion condensation effect, thereby inducing the aggregation of particles with opposite electric charges (negatively charged particles).

It should be understood that the ionizable groups can also be other functional groups with ionization capabilities.

Moreover, the molecular chain of the polyelectrolyte also contains at least one active group. The polyelectrolyte can react with the groups on the polymer chains of the crosslinked polymer through at least one active group to be bonded together (i.e., a chemical bond can be formed between the polyelectrolyte and the polymer chains of the crosslinked polymer), thereby allowing the plurality of polyelectrolytes to be grafted onto the three-dimensional network structure of the crosslinked polymer through chemical bonds.

Compared with conventional drug-loaded microsphere embolic agents, the polymeric material provided by the present disclosure, by grafting polyelectrolytes onto the three-dimensional network structure of the crosslinked polymer, can introduce more ionizable groups into the three-dimensional network structure of the crosslinked polymer, increasing both the overall number of ionizable groups in the polymeric material and the local density of the ionizable groups. This can enhance the adsorption amount and adsorption efficiency of the polymeric material toward particles with opposite electric charges. When the particles with opposite electric charges are drug particles, using the polymeric material provided by the present disclosure can increase the drug-loading capacity and shorten the drug-loading time.

In some exemplary embodiments, the plurality of ionizable groups on each of the polyelectrolytes are configured to form a local potential after ionization, inducing the aggregation of particles with opposite electric charges, which includes: each of the polyelectrolytes containing the plurality of ionizable groups, the plurality of ionizable groups forming a plurality of anionic groups after ionization, the plurality of anionic groups forming a local low potential capable of inducing the aggregation of positively charged particles. In the present disclosure, the plurality of anionic groups on each polyelectrolyte can be obtained after ionization of the plurality of ionizable groups on the polyelectrolyte. The ionizable groups can be acidic groups, which, after ionization, generate negatively charged groups (anionic groups); correspondingly, the polyelectrolyte, after ionization, becomes a polyanion. At this time, the polymeric material provided by the present disclosure can include: a crosslinked polymer; and a plurality of polyelectrolytes grafted onto the three-dimensional network structure of the crosslinked polymer through chemical bonds, where each of the polyelectrolytes contains a plurality of acidic groups (each acidic group forming one anionic group after ionization, and the plurality of acidic groups forming the plurality of anionic groups after ionization), and the plurality of anionic groups can form a local low potential through the counterion condensation effect to induce the aggregation of positively charged particles.

It should be understood that the ionizable groups can also be other types of functional groups capable of generating anions after ionization.

FIG. 2 shows a schematic diagram of the microscopic structure of a polymeric material provided by the present disclosure after ionization. The polymeric material provided by the present disclosure includes a crosslinked polymer and a plurality of polyelectrolytes. As shown in FIG. 2, taking gelatin methacrylate (GelMA) as an example of the crosslinked polymer, the three-dimensional network structure of the crosslinked polymer is formed by the interweaving of multiple polymer chains, and multiple types of groups are also formed on each polymer chain. These groups can exhibit different chemical activities depending on their chemical properties. The molecular chain of each polyelectrolyte contains a plurality of acidic groups, which, after ionization, can form a polyanion (the polyanion contains a plurality of negatively charged groups) and a plurality of cations (hydrogen ions). At this time, the plurality of negatively charged groups on the polyanion can adsorb positively charged particles (such as positively charged drugs) through electrostatic interactions, thereby achieving drug loading. Moreover, the molecular chain of the polyelectrolyte also contains at least one active group, and the polyelectrolyte can react with the groups on the polymer chains of the crosslinked polymer through at least one active group to be bonded together (i.e., a chemical bond can be formed between the polyelectrolyte and the polymer chains of the polymer), allowing the plurality of polyelectrolytes to be grafted onto the three-dimensional network structure of the crosslinked polymer through chemical bonds. Compared with conventional drug- loaded microsphere embolic agents, the polymeric material provided by the present disclosure, by grafting polyelectrolytes onto the three-dimensional network structure of the crosslinked polymer, can introduce more acidic groups into the three-dimensional network structure of the crosslinked polymer, increasing both the overall number of acidic groups in the polymeric material and the local density of the acidic groups. This can enhance the adsorption amount and adsorption efficiency of the polymeric material toward positively charged drugs, thereby increasing the drug-loading capacity and shortening the drug-loading time.

In the present disclosure, for each polyelectrolyte, since its molecular chain contains a plurality of acidic groups, the polyanion formed after ionization of the plurality of acidic groups contains a plurality of negatively charged groups. The aggregation of the plurality of negatively charged groups on one molecular chain can generate a counterion condensation effect, so that a local low potential can be formed near the polyelectrolyte, thereby allowing the polyelectrolyte to induce a large number of positively charged particles to aggregate toward itself through electrostatic interactions. In the polymeric material provided by the present disclosure, the plurality of polyelectrolytes are grafted onto the three-dimensional network structure of the crosslinked polymer through chemical bonds. Therefore, in the polymeric material, the negatively charged groups on the polyanions formed after ionization of the polyelectrolytes can be distributed on the three-dimensional network structure in a locally aggregated form (with relatively high local density).

Moreover, when the total amount of acidic groups is substantially the same, the denser the acidic groups in the polymeric material, the stronger the electrostatic adsorption capability after ionization. For example, when the total amount of acidic groups is substantially the same, the number of polyelectrolytes is inversely proportional to the number of acidic groups contained on each polyelectrolyte, which can include the following two situations: Class A polymeric material, where the number of polyelectrolytes is small, but each polyelectrolyte contains a large number of acidic groups (high local density); Class B polymeric material, where the number of polyelectrolytes is large, but each polyelectrolyte contains a small number of acidic groups (low local density). For the above two situations, due to the counterion condensation effect, Class A polymeric material exhibits a stronger ability to induce positively charged particles than Class B polymeric material, meaning that Class A polymeric material can adsorb a greater number of positively charged particles and has a faster adsorption rate. Therefore, compared with conventional drug-loaded microsphere embolic agents, even when the total amount of acidic groups is substantially the same, the polymeric material provided by the present disclosure, in which acidic groups are distributed in a locally aggregated form on the three-dimensional network structure of the polymer, can induce more positively charged particles to be electrostatically adsorbed and exhibit a faster electrostatic adsorption rate toward the positively charged particles, thereby increasing the drug-loading capacity while shortening the drug-loading time.

In some exemplary embodiments, the plurality of ionizable groups on each of the polyelectrolytes includes: each of the polyelectrolytes containing more than three ionizable groups.

In the present disclosure, for each polyelectrolyte in the polymeric material, the number of ionizable groups contained on the polyelectrolyte can be ≥3. For example, the number of ionizable groups can be 3, 4, 5, 6, 7, 8, 9, 10, 11, 12, 13, 14, 15, or any number greater than 15.

Taking the ionizable groups on the polyelectrolyte as acidic groups as an example, since the ionizable groups (acidic groups) on the polyelectrolyte can generate negatively charged groups and cations after ionization, when the polyelectrolytes of the present disclosure are in an ionized state, the polyelectrolytes form polyanions after ionization, and the number of negatively charged groups on the polyanion is the same as the number of ionizable groups contained on the polyelectrolyte. Therefore, the number of negatively charged groups on the polyanion can be ≥3; for example, the number of negatively charged groups can be 3, 4, 5, 6, 7, 8, 9, 10, 11, 12, 13, 14, 15, or any number greater than 15. At this time, the greater the number of ionizable groups contained on each polyelectrolyte, the higher the local density of the ionizable groups (corresponding to the higher local density of the negatively charged groups on the polyanion formed after ionization of the polyelectrolyte). In this case, the counterion condensation effect formed by the plurality of negatively charged groups is stronger, and the electrostatic adsorption ability of the polyanion toward positively charged particles is stronger.

Since the ionizable groups on the polyelectrolyte can also be other types of functional groups (such as basic groups, etc.), it should be understood that when the ionizable groups on the polyelectrolyte are basic groups, the greater the number of ionizable groups contained on each polyelectrolyte, the higher the local density of the ionizable groups (corresponding to the higher local density of the positively charged groups on the polycation formed after ionization of the polyelectrolyte). In this case, the counterion condensation effect formed by the plurality of positively charged groups is stronger, and the electrostatic adsorption ability of the polycation toward negatively charged particles is stronger.

In some exemplary embodiments, the ionizable groups include at least one of a carboxyl group, sulfonic acid group, sulfinic acid group, phosphoric acid group, phosphonic acid group, and hypophosphorous acid group.

In the present disclosure, the ionizable groups contained on the polyelectrolytes in the polymeric material can be of a single type or multiple types. Specifically, for one polyelectrolyte, the plurality of ionizable groups it contains can all be of one type, such as all being carboxyl groups, sulfonic acid groups, sulfinic acid groups, phosphoric acid groups, phosphonic acid groups, or hypophosphorous acid groups; or, for one polyelectrolyte, the plurality of ionizable groups it contains can be of two or more types, such as a polyelectrolyte containing both carboxyl groups and sulfonic acid groups. For the case where a polyelectrolyte contains two or more types of acidic groups, the specific possible combinations are not enumerated herein.

For all the polyelectrolytes in the polymeric material, the types of ionizable groups they contain depend on the types of ionizable groups contained in each polyelectrolyte. For example, when each polyelectrolyte contains only one type of ionizable group, and all the polyelectrolytes contain the same type of ionizable group, the types of ionizable groups contained in all the polyelectrolytes in the polymeric material are one; when each polyelectrolyte contains only one type of ionizable group, but two or more polyelectrolytes contain different types of ionizable groups, the types of ionizable groups contained in the polymeric material are greater than one; when each polyelectrolyte contains more than one type of ionizable group, the types of ionizable groups contained in the polymeric material are greater than one.

The type of polyelectrolyte can determine the type of ionizable groups contained in the polyelectrolyte; conversely, the type of ionizable groups contained in the polyelectrolyte can determine the type of polyelectrolyte. In some embodiments, the polyelectrolyte can include at least one of polyacrylic acid, carboxymethyl cellulose, carboxymethyl chitosan, sodium alginate, G4 dendrimer, polyglutamic acid, and polyaspartic acid.

Specifically, the polyelectrolytes in the polymeric material of the present disclosure can be any one of the above. For example, the polyelectrolyte can be polyacrylic acid, and a plurality of polyacrylic acid molecules can be grafted through chemical bonds onto different positions of the three-dimensional network structure of the crosslinked polymer, thereby forming the polymeric material.

The polyelectrolytes in the polymeric material of the present disclosure can also be any combination of two or more of the above. For example, the polyelectrolytes can include both polyacrylic acid and carboxymethyl cellulose, where at least one polyacrylic acid can be grafted through chemical bonds onto different positions of the three-dimensional network structure of the polymer, and at least one carboxymethyl cellulose can also be grafted through chemical bonds onto different positions of the three-dimensional network structure of the polymer, thereby forming the polymeric material. For the case where the types of polyelectrolytes in the polymeric material are two or more, the specific possible combinations are not enumerated herein.

In some exemplary embodiments, the crosslinked polymer can be a degradable crosslinked polymer. In the present disclosure, the crosslinked polymer has degradability, and during the degradation process, the macromolecular chains forming the three-dimensional network structure undergo cleavage. After the polymeric material is loaded with a drug (thus forming a drug-loading material), the cleavage of the macromolecular chains during the degradation of the crosslinked polymer can facilitate drug release; moreover, as the crosslinked polymer is completely degraded, the drug loaded in the polymeric material can be fully released, thereby improving drug utilization. In addition, because the crosslinked polymer is degradable, as it degrades in vivo, the drug particles loaded on the polymeric material are continuously released, allowing the drug particles at the location of the drug-loading material (i.e., the embolization site) to be maintained at a stable concentration level, thereby improving drug utilization while ensuring that the drug continuously exerts a stable therapeutic effect.

In some exemplary embodiments, the release rate of the loaded drug can be regulated by controlling the degradation rate of the crosslinked polymer, thereby achieving a long-acting drug sustained-release effect.

For polymeric materials prepared using non-degradable crosslinked polymers, when the polymeric material is loaded with a drug and applied in vivo, the non-degradability of the crosslinked polymer can cause a sustained immune response in the body. In the polymeric material provided by the present disclosure, a degradable crosslinked polymer is used. When the polymeric material provided by the present disclosure is loaded with a drug and applied in vivo, the immune response generated in the body gradually subsides as the crosslinked polymer degrades. In the present disclosure, since the polymeric material can be used for drug loading, the effect of the polymeric material on the organism can be taken into account during application, and a crosslinked polymer with good biocompatibility can be selected as the raw material of the polymeric material, thereby reducing or avoiding the immune response in the body.

In some exemplary embodiments, the degradable crosslinked polymer includes at least one of crosslinked-modified gelatin, crosslinked-modified chitosan, crosslinked-modified hyaluronic acid, crosslinked-modified agarose, crosslinked- modified chondroitin sulfate, crosslinked-modified starch, and crosslinked-modified cellulose.

Having a three-dimensional network structure is a fundamental structural feature of the crosslinked polymer. The three-dimensional network structure of the crosslinked polymer is generally formed by crosslinking modification of the precursor (including physical crosslinking, chemical crosslinking, and biological crosslinking). Physical crosslinking, also called non-covalent crosslinking, is generally formed through weak interactions such as hydrogen bonding, hydrophobic interactions, electrostatic interactions, and host-guest recognition; chemical crosslinking, also called covalent crosslinking, is formed by generating new chemical bonds through chemical reactions, including classical click chemistry reactions, Diels-Alder (DA) addition reactions, Michael addition, and the formation of Schiff bases, disulfide bonds, boronate ester bonds, and coordination bonds. Biological crosslinking mainly refers to enzyme-mediated crosslinking reactions. For example, chondroitin sulfate contains a large number of carboxyl groups, sulfate groups, and adjacent hydroxyl groups on its sugar chains, which not only provide abundant reaction sites for chemical modification but also provide potential active groups for physical and enzymatic crosslinking.

Gelatin, chitosan, hyaluronic acid, agarose, chondroitin sulfate, starch, and cellulose can all be crosslink-modified to form crosslinked polymers with a three-dimensional network structure. Since gelatin, chitosan, hyaluronic acid, agarose, chondroitin sulfate, starch, and cellulose are all natural polymers, the crosslinked polymers obtained through crosslink modification have good biocompatibility and biodegradability.

For example, gelatin (Gelatin) is a natural polymer material obtained by hydrolyzing collagen, and it has advantages such as biodegradability, good biocompatibility and gelation, low cost, and non-toxicity, making it widely used in the food and pharmaceutical fields. Gelatin is composed of polypeptide chains of various amino acids and has the same repeating sequence as collagen, namely Gly-X-Y, where Gly is glycine, and X and Y represent any amino acid other than glycine.

The electrostatic repulsion between proline and hydroxyproline in the gelatin molecular chain promotes the formation of left-handed α-helices, which play an important role in stabilizing the gelatin structure. In addition, the gelatin molecular chain contains active groups such as amino, carboxyl, and hydroxyl groups. Currently, gelatin used for embolic agents is often temporary, such as the clinically used Gelatin Sponge, Gelfoam, and OptiSphere, all of which can be reabsorbed by the body. The gelatin molecular backbone retains short peptides such as arginine-glycine-aspartic acid (RGD) and matrix metalloproteinase (MMP) peptides, providing good cell adhesion and degradability. By using specific crosslinking reagents to form crosslinked structures in gelatin or catalyzing reactions of groups on gelatin, a self-crosslinked structure can be formed, i.e., crosslink-modified gelatin obtained through crosslink modification has a three-dimensional network structure. In the present disclosure, when the crosslinked polymer is crosslink-modified gelatin, the polyelectrolytes can be grafted onto the three-dimensional network structure of the crosslinked polymer via double-bond crosslinking. The polyelectrolytes can also be grafted through reactions between their other active groups and the amino, hydroxyl, carboxyl, and other groups on the gelatin backbone.

Chitosan molecular chains contain a large number of free amino (-NH₂), hydroxyl (-OH), N-acetyl (-CO-NH₂), and other reactive functional groups, and the amino groups of chitosan can attract anions through electrostatic interactions after ionization. Chitosan can be modified through crosslinking. In the crosslinking reaction, the chitosan molecular chain first reacts with a crosslinking agent to form an intermediate product, and then under certain conditions, a three-dimensional network structure of chitosan is formed. The crosslinking agent can be selected from at least one of glutaraldehyde, epichlorohydrin, ethylene glycol diglycidyl ether, and tripolyphosphate.

Hyaluronic acid (HA) is a natural mucopolysaccharide that exhibits unique viscoelasticity, excellent biological compatibility, and degradability due to its high molecular weight. By chemically modifying and crosslinking hyaluronic acid to form a three-dimensional network structure, its mechanical strength and stability can be enhanced.

Agarose, chondroitin sulfate, starch, cellulose, and the like can be crosslink-modified according to the types of functional groups on their molecular chains to form their respective three-dimensional network structures, which are not enumerated herein. Moreover, when the crosslinked polymer is crosslink-modified chitosan, hyaluronic acid, agarose, chondroitin sulfate, starch, cellulose, or the like, the polyelectrolytes can also be grafted onto the three-dimensional network structure of the crosslinked polymer via double-bond crosslinking, and the polyelectrolytes can be grafted through reactions according to the types of reactive groups contained in the three-dimensional network structure of the crosslinked polymer.

In the polymeric material provided by the present disclosure, the weight fractions of the crosslinked polymer and the polyelectrolytes can be adjusted according to actual needs. Generally, when the weight of the crosslinked polymer remains constant, the greater the weight fraction of the same polyelectrolyte, the greater the number of negatively charged groups (which can adsorb positively charged particles) obtained after ionization. In some exemplary embodiments, by mass percentage, the crosslinked polymer accounts for 1-30% of the total mass, and the plurality of polyelectrolytes account for 0.2-30% of the total mass. For example, by mass percentage, the crosslinked polymer can account for 1%, 2%, 3%, 4%, 5%, 6%, 7%, 8%, 9%, 10%, 11%, 12%, 13%, 14%, 15%, 16%, 17%, 18%, 19%, 20%,21%, 22%, 23%, 24%, 25%, 26%, 27%, 28%, 29%, 30%, or any value between any two of the above percentages of the total mass of the polymeric material; the polyelectrolytes can account for 0.2%, 0.3%, 0.4%, 0.5%, 0.6%, 0.7%, 0.8%, 0.9%, 1%, 2%, 2.2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, or any value between any two of the above percentages of the total mass of the polymeric material.

It should be noted that in the polymeric material 100 of the present disclosure, in addition to the crosslinked polymer 120 and the polyelectrolytes 142, water molecules present in the three-dimensional network structure of the crosslinked polymer are also included. The mass percentage of water in the polymeric material can reach over 70%. Moreover, in the polymeric material 100 of the present disclosure, the mass percentages of the crosslinked polymer 120 and the polyelectrolytes 142 are also related to the types of the crosslinked polymer and polyelectrolytes, their respective molecular weights, the number of active groups contained in each, and other parameters.

In the polymeric material 100 of the present disclosure, the connection between the crosslinked polymer 120 and the polyelectrolytes 142 can be either direct or indirect. Direct connection can be understood as the reactive groups on the polyelectrolyte molecular chain directly reacting with the groups on the crosslinked polymer molecular chain to form a bond. Indirect connection can be understood as the reactive groups on the polyelectrolyte molecular chain being linked to the molecular chain of the crosslinked polymer through an intermediate substance 144, which contains at least two functional groups. For ease of description, one of the two functional groups is referred to as the first functional group, and the other is referred to as the second functional group. The first functional group can be used to connect with the reactive groups on the polyelectrolyte molecular chain, and the second functional group can be used to connect with the reactive groups on the crosslinked polymer molecular chain. For example, in some embodiments, the polymeric material 100 further includes a bridging agent 144 positioned between the polyelectrolytes 142 and the crosslinked polymer 120, for connecting the polyelectrolytes 142 to the crosslinked polymer 120. The bridging agent 144 is an intermediate material capable of simultaneously connecting the polyelectrolyte molecular chain and the crosslinked polymer molecular chain.

In the present disclosure, depending on the chemical reactivity of the bridging agent itself, there are various possible relative positions of the first and second functional groups on the bridging agent 144. When the first and second functional groups on the bridging agent 144 are relatively far apart (which can also be understood as the first and second functional groups being located at the two ends of the bridging agent molecular chain), during the process in which the polyelectrolytes 142 are grafted onto the crosslinked polymer molecular chain via the bridging agent 144, if the molecular chain of the bridging agent 144 is relatively long, the polyelectrolyte molecular chain can extend away from the main body of the three-dimensional network structure of the crosslinked polymer 120 due to the spacing effect of the bridging agent 144. This can reduce steric hindrance between the polyelectrolytes 142 and the crosslinked polymer 120. Under this circumstance, after ionization of the acidic groups on the polyelectrolytes 142, more negatively charged groups can be exposed, thereby enabling the adsorption of a greater number of positively charged particles through electrostatic interactions.

In some exemplary embodiments, the bridging agent 144 includes at least two functional groups, and the bridging agent 144 connects the polyelectrolytes 142 to the crosslinked polymer 120 through the at least two functional groups, where each of the at least two functional groups includes any one of amino, carboxyl, aldehyde, thiol, carbon-carbon double bond, carbon-carbon triple bond, acryloyl, isobutyryl, azido, epoxy, vinyl sulfone, succinimide, biotin, dibenzocyclooctyne, di(p-nitrophenyl) carbonate, or norbornene.

In the present disclosure, the bridging agent 144 connects to the polyelectrolytes 142 and the crosslinked polymer 120 via the functional groups it contains, thereby achieving an indirect connection between the polyelectrolytes 142 and the crosslinked polymer 120 through the bridging agent 144. As described above, to ensure that the bridging agent 144 can simultaneously connect the polyelectrolyte molecular chain and the crosslinked polymer molecular chain, the bridging agent 144 includes at least two functional groups. In the present disclosure, the number of functional groups on the bridging agent 144 can be 2, 3, 4, 5, 6, or more.

When the bridging agent 144 has two functional groups (namely the first functional group and the second functional group), the first functional group can be connected to the polyelectrolytes 142, and the second functional group can be connected to the crosslinked polymer 120. In the present disclosure, the first and second functional groups can be of the same type or of different types. For example, when the first and second functional groups are the same, they can each be any one of amino, carboxyl, aldehyde, thiol, carbon-carbon double bond, carbon-carbon triple bond, acryloyl, isobutyryl, azido, epoxy, vinyl sulfone, succinimide, biotin, dibenzocyclooctyne, di(p-nitrophenyl) carbonate, or norbornene. When the first and second functional groups are of different types, if the first functional group is amino, the second functional group can be any other functional group except amino (such as carboxyl, aldehyde, thiol, carbon-carbon double bond, carbon-carbon triple bond, acryloyl, isobutyryl, azido, epoxy, vinyl sulfone, succinimide, biotin, dibenzocyclooctyne, di(p-nitrophenyl) carbonate, or norbornene). Other possible combinations of different types of first and second functional groups are not enumerated herein.

When the bridging agent 144 has three functional groups (namely the first functional group, the second functional group, and the third functional group), if the first functional group is connected to the polyelectrolytes 142 and the second functional group is connected to the crosslinked polymer 120, in this case, the third functional group can serve an auxiliary role by connecting to the polyelectrolytes 142; or the third functional group can serve an auxiliary role by connecting to the crosslinked polymer 120. Similarly, when the bridging agent 144 has four or more functional groups, the functional groups on the bridging agent 144 can be connected respectively to the polyelectrolytes 142 and the crosslinked polymer 120, so that the polyelectrolytes 142 and the crosslinked polymer 120 form an indirect connection through the bridging agent 144.

It should be noted that the type of functional groups on the bridging agent 144 and the number of functional groups are two different concepts. In the present disclosure, when the number of functional groups on the bridging agent 144 is 2, 3, 4, 5, or more than 6, the functional groups can be of the same type or of different types.

In the present disclosure, the bridging agent 144 can be a small molecule compound or a high-molecular polymer. For example, the bridging agent 144 can be a polyethylene glycol derivative with one or more functional groups, where the functional groups of the polyethylene glycol derivative can include at least one of amino, carboxyl, aldehyde, thiol, carbon-carbon double bond, carbon-carbon triple bond, acryloyl, isobutyryl, azido, epoxy, vinyl sulfone, succinimide, biotin, dibenzocyclooctyne, di(p-nitrophenyl) carbonate, or norbornene. The bridging agent 144 can also be other small molecule compounds or high-molecular polymers containing at least one of the above types of functional groups.

In some exemplary embodiments, the molecular design of the bridging agent 144 has a critical impact on the performance of the final material. For example, when the bridging agent is a polyethylene glycol (PEG) chain bearing an amino group (NH₂-, serving as the first functional group) at one terminus and a methacrylate group (serving as the second functional group) at the other terminus (i.e., NH₂-PEG-MA), its coupling process can be described as follows: First, the amino group of the bridging agent 144 reacts with the carboxyl group of polyelectrolyte 142 (e.g. polyacrylic acid, PAA) at 1:1 molar ratio at the presence of 1-ethyl-3-(3-dimethylaminopropyl)carbodiimide (EDC) and N-hydroxysuccinimide (NHS), producing bridging agent-modified polyelectrolyte 140 (i.e. mono-methacrylate PEGylated PAA, PAA-PEGMA). Subsequently, the methacrylate group at the other side of the bridging agent 144 in PAA-PEGMA reacts with the methacrylate group in the precursor of the crosslinked polymer 120 (e.g. gelatin methacrylate, GelMA) at the presence of photoinitiator (e.g. lithium phenyl(2,4,6-trimethylbenzoyl) phosphinate, LAP) under UV irradiation in a pH 7–9 buffer, forming crosslinked polymer network 120 and simultaneously anchoring the bridging agent-modified polyelectrolyte 140 to the crosslinked polymer network 120. In this process, the chain length of the PEG bridging agent (for example, a molecular weight of 2000 Da corresponds to approximately 45 ethylene glycol units and an extended length of about 15 nm) effectively increases the distance between the polyelectrolyte and the network backbone, significantly reducing steric hindrance.

The types and relative positions of the functional groups on the bridging agent 144 determine the chemical selectivity of the grafting reaction and the conformation of the polyelectrolyte chains. In addition to the heterobifunctional PEG example described above, the bridging agent 144 may also be a small molecule. For instance, 1-ethyl-3-(3-dimethylaminopropyl)carbodiimide (EDC) used in combination with N-hydroxysulfosuccinimide (Sulfo-NHS) can in situ activate the carboxyl groups on the polyelectrolyte 142 (e.g., polyglutamic acid), enabling them to react with the amino groups on the crosslinked polymer 120 (e.g., a gelatin network), in which case the EDC/Sulfo-NHS system itself serves as a “transient” bridging agent. In some exemplary embodiments, the bridging agent 144 may be a multi-arm PEG derivative bearing multiple epoxide groups (e.g., four-arm PEG-epoxy). Its epoxide groups (serving simultaneously as the first and second functional groups) can undergo ring-opening reactions under basic conditions with both the amino groups on the crosslinked polymer 120 (e.g., chitosan) and the carboxyl groups on the polyelectrolyte 142 (e.g., carboxymethyl cellulose), forming branched connection points. When the bridging agent 144 itself is a polymer with terminal functional groups (for example, an α-amino-ω-carboxyl polystyrene-b-polyethylene glycol block copolymer), its relatively long hydrophobic polystyrene segment (assumed to have been pre-incorporated into the crosslinked network as part of the structure) and its hydrophilic PEG segment can form micelle-like structures. The polyelectrolyte 142 (e.g., sodium polystyrene sulfonate, PSS) is then grafted onto the corona region of the micelles through EDC-mediated coupling between the carboxyl group at the PEG terminus and the amino groups modified onto the PSS chain. This architecture allows the PSS chains to extend fully into the aqueous phase, with their sulfonate negative charges experiencing minimal steric obstruction from the crosslinked network. As a result, the adsorption capacity for cationic dyes (such as methylene blue) can be higher than that achieved via direct linkage methods.

In the present disclosure, the bridging agent 144 can first react with the polyelectrolytes 142, thereby grafting onto the polyelectrolyte molecular chain; after the bridging agent 144 is grafted onto the polyelectrolyte molecular chain, it then reacts with the crosslinked polymer 120, so that the polyelectrolytes 142 and the crosslinked polymer 120 are indirectly connected. The bridging agent 144 can also first react with the crosslinked polymer 120, thereby grafting onto the crosslinked polymer molecular chain; after the bridging agent 144 is grafted onto the crosslinked polymer molecular chain, it then reacts with the polyelectrolytes 142, so that the crosslinked polymer 120 and the polyelectrolytes 142 are indirectly connected. The bridging agent 144 can also simultaneously react with both the polyelectrolytes 142 and the crosslinked polymer 120, thereby indirectly connecting the crosslinked polymer 120 and the polyelectrolytes 142.

The coupling sequence of the bridging agent 144 can be optimized according to process requirements. For example, a bridging agent 144 containing a carbon–carbon double bond (such as glycidyl methacrylate, GMA) is first grafted onto the precursor of the crosslinked polymer 120 (e.g., a polyvinyl alcohol, PVA, solution) through a ring-opening reaction between its epoxide group (the second functional group) and a portion of the hydroxyl groups on the PVA chains in the presence of a catalyst. Subsequently, during or after the formation of the chemically crosslinked PVA network (for example, via addition of glutaraldehyde as a crosslinker), the carbon–carbon double bond (the first functional group) on GMA undergoes copolymerization, initiated by a thermal initiator (such as ammonium persulfate), with a double-bond-containing polyelectrolyte 142 (for instance, a copolymer of sodium polyacrylate and acryloyloxyethyl trimethyl ammonium chloride, partially grafted with acrylate groups). Through this process, the polyelectrolyte is indirectly anchored to the network via covalent bonds. This “graft-bridging-agent first, graft-polyelectrolyte later” sequence facilitates control over the grafting density and uniform distribution of the polyelectrolyte. Conversely, if the polyelectrolyte 142 (such as sodium alginate) is first oxidized with sodium periodate to generate aldehyde groups (serving as its reactive groups), and then reacts with one hydrazide group (the first functional group) of a bridging agent 144 such as adipic dihydrazide (ADH) to form a hydrazone bond, the resulting polyelectrolyte–ADH intermediate can subsequently couple to the remaining aldehyde groups on another pre-formed crosslinked polymer 120 network (for example, a gel formed by crosslinking oxidized sodium alginate with Ca²⁺) through the second hydrazide group on ADH. This achieves a “modify the polyelectrolyte first, then connect to the network” strategy. These two sequences provide flexibility in material synthesis, allowing adaptation to different processing conditions (such as aqueous-phase or organic-phase reactions) and avoiding undesired side reactions between functional groups.

It should be understood that, in addition to the types of functional groups listed above, the bridging agent 144 may also include other functional groups that do not have the function of connecting the polyelectrolytes 142 and the crosslinked polymer 120.

In the present disclosure, the introduction of a bridging agent provides a modular and precisely designed interfacial coupling strategy. Compared with direct linkage between the polyelectrolyte and the crosslinked polymer, the use of a bridging agent not only effectively alleviates steric hindrance through its molecular chain length—thereby allowing the active sites of the polyelectrolyte to be fully exposed and significantly enhancing material functionalities (such as ion adsorption capacity and rate)—but, more importantly, enables efficient and directional covalent coupling in diverse chemical environments through the selectivity and versatility of its functional groups. This greatly improves the flexibility and general applicability of material synthesis. Accordingly, the bridging agent serves not merely as a “bridge” for achieving indirect connection, but as a key tool for actively designing the microscopic architecture and optimizing the performance of the material, providing a solid foundation for the customized development of high-performance composite polymer materials.

In a second aspect, the present disclosure provides a method for preparing a polymeric material, including: mixing a crosslinking precursor, a crosslinking agent, and polyelectrolytes, and then performing a crosslinking polymerization reaction under external condition stimulation to form the polymeric material.

In the present disclosure, the polymeric material can be prepared by a photochemical crosslinking method, a conventional chemical crosslinking method (thermal crosslinking method), or a combination of the photochemical crosslinking method and the conventional chemical crosslinking method.

For the process of preparing the polymeric material by a photochemical crosslinking method, a microfluidics-assisted photochemical crosslinking method can be used to prepare polymeric materials with specific shapes and sizes. The method for preparing by microfluidics-assisted photochemical crosslinking can include the following step:

(1) Preparing an aqueous solution: PBS buffer can be used as the solvent, and then the crosslinking precursor solution, polyelectrolyte solution, and photoinitiator are mixed to obtain the aqueous solution;

Depending on the type of reactive intermediates generated under light irradiation, the photoreactive groups can be selected from photoinitiators of azine type, carbene type, carbocation type, or free radical type. For example, free radical photoinitiators can include at least one of the following: lithium phenyl(2,4,6-trimethylbenzoyl)phosphinate (LAP), diphenyl(2,4,6-trimethylbenzoyl)phosphine oxide (TPO), 2,4,6-trimethylbenzoylphenyl phosphinate ethyl ester (TPO-L), 2-methyl-1-[4-(methylthio)phenyl]-2-morpholino-1-propanone (907), 2-isopropylthioxanthone (ITX), 1-hydroxycyclohexyl phenyl ketone (184), 2-hydroxy-2-methylpropiophenone (1173), 2,2-dimethoxy-2-phenylacetophenone (BDK), 2-benzoylbenzoic acid methyl ester (OMBB), benzophenone (BP), 4-chlorobenzophenone (CBP), 4-phenylbenzophenone (PBZ), 2-phenylbenzyl-2-dimethylamino-1-(4-morpholinophenyl)butanone (369), 1,1’-(methylene bis-4,1-phenylene)bis[2-hydroxy-2-methyl-1-propanone] (127), 4-(dimethylamino)benzophenone ethyl ester (EDB), isooctyl 4-(dimethylamino)benzoate (EHA), 4-methylbenzophenone (MBZ), 2,4-diethylthioxanthone-9-one (DETX), 2-(4-methylbenzyl)-2-(dimethylamino)-1-(4-morpholinophenyl)-1-butanone (379), 4-benzoyl-4’-methyl-diphenyl sulfide (BMS), 2,2’-(o-chlorophenyl)-4,4’,5,5’-tetraphenyl-1,2’-biimidazole (BCIM), 2,2’-(o- chlorophenyl)-4,4’,5,5’-tetraphenyl-1,2’-biimidazole (OXE01), 1-[4-(4-benzoylphenylthio)phenyl]-2-toluenesulfonyl-2-methyl-1-propanone (1001M), 2-hydroxy-4’-(2-hydroxyethoxy)-2-methylpropiophenone (Irgacure 2959), benzoylformate methyl ester (MBF), or 2-ethylanthraquinone (EAQ).

Cationic photoinitiators can include at least one of the following: arylthio/iodonium salts, triphenylhexafluorophosphate sulfonium salts, diphenyliodonium hexafluorophosphate (810), 4-(phenylthio)phenyl diphenylsulfonium salt, tris[4-[(4-acetylphenyl)thio]phenyl]-hexafluorophosphate (Irgacure 209), 4-dodecyloxyphenyl diphenylsulfonium hexafluoroantimonate (SOC 10), 9-[4-(2-hydroxyethoxy)phenyl]thianthrene sulfonium hexafluorophosphate (Esacure 1187), 10-(4-biphenyl)-2-isopropylthioxanthone sulfonium hexafluorophosphate (Omnicat 550), [7-(1-methylpropyl)-9-oxo-9H-thioxanthene-2-yl]bis(4-methylphenyl)sulfonium hexafluoroantimonate (PCI 061T), bis[(4-diphenylsulfonium)phenyl] sulfide bis-hexafluorophosphate (UV 6992), 4-(phenylthio)phenyl diphenylsulfonium salt, 4,4-bis(thianthren-9-yl)biphenyl hexafluorophosphate, S,S’-(thiodi-4,1-phenylene)bis[S,S-bis[4-(2-hydroxyethoxy)phenyl]sulfonium hexafluorophosphate] (SP 150), (4-hydroxyphenyl)methyl(benzyl)hexathioium hexafluorophosphate (PHS SI 100L), 4-acetoxyphenyl dimethylsulfonium hexafluoroantimonate (Sanaid SI 150), (4-hydroxyphenyl)methyl[(2-methylphenyl)methyl]sulfonium hexafluoroantimonate (Sanaid 80L), dodecylmethyl(2-oxo-2-phenylethyl)sulfonium hexafluoroantimonate, diphenyliodonium hexafluorophosphate (Photoinitiator 810), bis(4-methylphenyl)iodonium hexafluorophosphate, bis(4-dodecylphenyl)iodonium hexafluorophosphate, bis(4-isopropylphenyl)iodonium hexafluorophosphate, bis(4-tert-butylphenyl)iodonium hexafluorophosphate, (4-methylphenyl)[4-(2-methylpropyl)phenyl]iodonium hexafluorophosphate (Irgacure 250), and 4-octyloxyphenyl iodonium hexafluoroantimonate (UVACURE 1600).

(2) Selecting an oil phase: pure soybean oil (CAS: 8001-22-7), paraffin oil, or other vegetable oils or mineral oils can be used.

(3) Preparing a collection solution: 1X PBS-10X PBS can be used as the collection solution;

(4) Selecting ultraviolet light with a suitable wavelength range and power;

(5) Building an experimental system: for example, the experimental system can be built as follows: use an infrared lamp, an incubator, an oven, and other heat sources to ensure that the crosslinking precursor solution remains in a liquid state in both the syringe and the flow channel; draw the oil-phase solution and the aqueous-phase solution separately into syringe barrels, connect the syringe needle heads to silicone tubes, fit small steel tubes onto the other ends of the silicone tubes, and insert the small steel tubes into the cross-shaped inlet to achieve injection; additionally, mount the syringe barrels on a micro-injection pump, which controls the flow rates of the two solutions; allow the aqueous-phase solution and the oil-phase solution entering the microfluidic chip to meet at the intersection of the “cross”-shaped structure; since the oil phase and the aqueous phase are immiscible, and the oil phase is set to have a relatively high flow rate, the photocrosslinkable aqueous-phase solution is sheared into droplets of a certain length; after droplet formation, the droplets are solidified in the channel (ultraviolet irradiation of the droplets in the flow channel) and discharged at the end of the straight channel, with the outlet end of the microfluidic chip immersed in a beaker filled with the collection solution; heating and stirring the collection solution can prevent particle aggregation.

(6) Collecting a particulate polymer material: centrifuge the collection solution, separate the particulate polymer material at the bottom, wash it, and store it. For the process of preparing polymer materials using an ordinary chemical crosslinking method (for example, an emulsion preparation method), or a combination of a photochemical crosslinking method and an ordinary chemical crosslinking method, considering the widespread use of commercial microspherical embolic agents, glutaraldehyde, paraformaldehyde, formaldehyde, and other crosslinking agents may also be used for preparation, and the crosslinking agents can adjust the crosslinking degree of the crosslinked polymer.

For the process of preparing polymer materials using a photochemical crosslinking method, an ordinary chemical crosslinking method (thermal crosslinking method), or a combination of a photochemical crosslinking method and an ordinary chemical crosslinking method, polyelectrolytes can also be grafted onto the three-dimensional network structure of the crosslinked polymer by introducing bridging agents (such as polyethylene glycol derivatives, N-hydroxysuccinimide, 1-ethyl-(3-dimethylaminopropyl) carbodiimide, acrylamide, etc.).

Therefore, in some embodiments, the method for preparing the polymer material further includes: mixing the crosslinking precursor, crosslinking agent, polyelectrolyte, and bridging agent, and then performing a crosslinking polymerization reaction under external stimulation. The polymer material prepared by the above method contains the bridging agent. In the present disclosure, polyethylene glycol derivatives and the like can serve as double-bond introducing agents for the polyelectrolyte during the photochemical crosslinking preparation of the polymer material, and further undergo photochemical crosslinking with the crosslinked polymer having double bonds.

In some exemplary embodiments, taking the preparation of polymer materials using an ordinary chemical crosslinking method as an example, in which glutaraldehyde is used as the crosslinking agent during the preparation, and a bridging agent is introduced, the preparation method may include the following steps:

(1) Preparing a polyelectrolyte with a single-chain acidic group content of 3 or more into an aqueous solution;

(2) Adding a bridging agent to the aqueous solution obtained in step (1) to form a reaction solution, and freeze-drying the reaction solution for storage;

(3) Dissolving a crosslinking precursor in a hydrochloric acid solution to obtain a crosslinking precursor solution;

(4) Mixing the reaction solution obtained in step (2) with the crosslinking precursor solution obtained in step (3) to obtain a mixed solution, and controlling the mixed solution to have no solid precipitation;

(5) Preparing an oil phase: using soybean oil containing the crosslinking agent (glutaraldehyde) as the oil phase;

(6) Adding the mixed solution obtained in step (4) to the oil phase obtained in step (5), stirring to obtain a suspension, performing a crosslinking reaction under heating conditions, stopping stirring when the suspension becomes clear, and allowing the mixture to stand to wait for the polymer microspheres to settle, where the polymer microspheres have a size between 50 µm-1000 µm;

(7) After removing the oil phase, washing to remove the oil phase on the surface of the polymer microspheres, and separating the polymer microspheres by graded filtration to store polymer microspheres of different sizes separately.

In some exemplary embodiments, taking as an example the preparation of polymer materials by combining an ordinary chemical crosslinking method, where glutaraldehyde is used as the crosslinking agent during the preparation and a bridging agent is introduced, the preparation method may include the following steps:

(1) Preparing a polyelectrolyte with a single-chain acidic group content of 3 or more into an aqueous solution;

(2) Adding a bridging agent to the aqueous solution obtained in step (1) to form a reaction solution, and freeze-drying the reaction solution for storage;

(3) Dissolving a crosslinking precursor in a hydrochloric acid solution to obtain a crosslinking precursor solution;

(4) Mixing the reaction solution obtained in step (2) with the crosslinking precursor solution obtained in step (3) to obtain a mixed solution, and controlling the mixed solution to have no solid precipitation;

(5) Preparing an oil phase: using soybean oil containing the crosslinking agent (glutaraldehyde) as the oil phase;

(6) Adding the mixed solution obtained in step (4) to the oil phase obtained in step (5), stirring to obtain a suspension, performing a crosslinking reaction under ultraviolet irradiation, stopping irradiation when the suspension becomes clear, and

allowing the mixture to stand to wait for the polymer microspheres to settle, where the polymer microspheres have a size between 50 µm-1000 µm;

(7) After removing the oil phase, washing to remove the oil phase on the surface of the polymer microspheres, and separating the polymer microspheres by graded filtration to store polymer microspheres of different sizes separately.

In a third aspect, the present disclosure provides an embolic agent, and the embolic agent is made of the polymer material. The polymer material provided by the present disclosure can be used as an embolic agent. When the polymer material provided by the present disclosure is used as an embolic agent, the forming particle size of the polymer material can be controlled during the preparation process of the polymer material.

In some exemplary embodiments, the particle size of the embolic agent may be 50-1000 µm. For example, the particle size of the embolic agent may be 50 µm, 70 µm, 100 µm, 150 µm, 200 µm, 250 µm, 300 µm, 350 µm, 400 µm, 450 µm, 500 µm, 550 µm, 600 µm, 650 µm, 700 µm, 750 µm, 800 µm, 850 µm, 900 µm, 950 µm, 1000 µm, or a value between any two of the above particle sizes.

In some exemplary embodiments, the particle size of the embolic agent may also be 100-300 µm. For example, the particle size of the embolic agent may be 100 µm, 110 µm, 120 µm, 130 µm, 140 µm, 150 µm, 160 µm, 170 µm, 180 µm, 190 µm, 200 µm, 210 µm, 220 µm, 230 µm, 240 µm, 250 µm, 260 µm, 270 µm, 280 µm, 290 µm, 300 µm, or a value between any two of the above particle sizes.

In a fourth aspect, the present disclosure provides a drug-loading material, the drug-loading material including: a polymer material and a plurality of drug particles, where the polymer material includes a crosslinked polymer and a plurality of polyions, the plurality of polyions being grafted onto the three-dimensional network structure of the crosslinked polymer through chemical bonds, and each of the polyions containing a plurality of charged groups, the plurality of charged groups forming a local potential to induce aggregation of drug particles having an opposite charge; the plurality of drug particles being adsorbed onto the polymer material through electrostatic interaction.

The drug-loading material provided by the present disclosure can be understood as the polymer material after drug loading, and therefore the drug-loading material may include the polymer material and a plurality of drug particles. During the process of drug loading using the polymer material, for each polyelectrolyte included in the polymer material, the multiple ionizable groups on the polyelectrolyte ionize to form multiple polyions. If the ionizable groups are acidic groups, the resulting polyions are polyanions, and correspondingly, each polyanion contains multiple negatively charged groups (anionic groups). The multiple negatively charged groups can form a local low potential through a counter-ion condensation effect, thereby attracting positively charged drug particles. If the ionizable groups are basic groups, the resulting polyions are polycations, and correspondingly, each polycation contains multiple positively charged groups. The multiple positively charged groups can form a local high potential through a counter-ion condensation effect, thereby adsorbing negatively charged drug particles.

Compared with conventional drug-loaded microsphere embolic agents, the drug-loading material provided by the present disclosure can graft polyelectrolytes onto the three-dimensional network structure of the crosslinked polymer, thereby introducing more ionizable groups into the three-dimensional network structure of the crosslinked polymer and increasing the total number of ionizable groups carried by the polymer material. As a result, the polymer material can achieve a higher adsorption amount and adsorption efficiency for drug particles with opposite charges, meaning that the drug-loading material provided in the present disclosure can have a higher drug-loading capacity and a shorter drug-loading time.

In some exemplary embodiments, the drug-loading material includes a polymer material and a plurality of drug particles, where the polymer material includes a crosslinked polymer and a plurality of polyanions, the plurality of polyanions being grafted onto the three-dimensional network structure of the crosslinked polymer through chemical bonds, and each of the polyanions containing a plurality of negatively charged groups, the plurality of negatively charged groups forming a local low potential to induce aggregation of positively charged particles; the plurality of drug particles include a plurality of positively charged drug particles, and the plurality of positively charged drug particles are adsorbed onto the polymer material through electrostatic interaction.

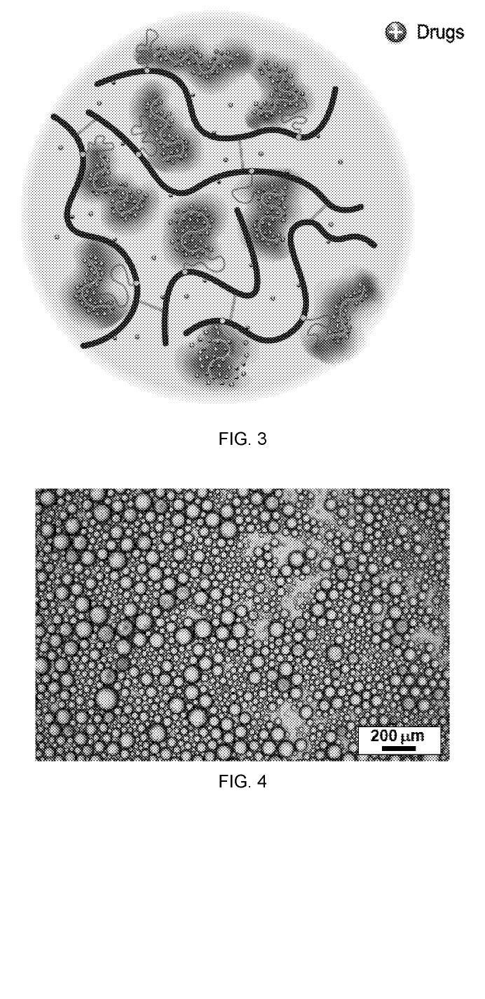

FIG. 3 illustrates a schematic diagram of the microstructure of a drug-loading material provided by the present disclosure. The polyions in FIG. 3 are polyanions. In combination with FIGS. 2 and 3, the drug-loading material provided by the present disclosure includes positively charged drug particles and the polymer material shown in FIG. 2, the drug particles can be locally aggregated and adsorbed onto the plurality of polyanions of the polymer material through electrostatic interaction. At this time, the positively charged drug particles are adsorbed onto the plurality of polyanions (or ionized polyelectrolytes) contained in the polymer material through electrostatic interaction, such that the drug particles mainly exhibit a locally aggregated state.

In addition, the positively charged drug particles can also be adsorbed onto the negatively charged groups carried by the crosslinked polymer itself through electrostatic interaction. Since the number of negatively charged groups carried by the crosslinked polymer itself is much lower than the number of negatively charged groups contained in the polyanions, and the negatively charged groups carried by the crosslinked polymer itself are randomly distributed (making it difficult to form a counter-ion condensation effect), the number of drug particles directly adsorbed onto the negatively charged groups of the crosslinked polymer itself is relatively small and they are in a random distribution state.

The properties of the drug particles can determine the therapeutic effect of the drug-loading material provided by the present disclosure. Depending on the type of drug particles adsorbed onto the polymer material through electrostatic interaction, the drug-loading material can exhibit different therapeutic effects. In the present disclosure, the drug particles can be selected from various drug molecules that can carry a charge after ionization. For example, in some embodiments, the drug particles may include at least one of doxorubicin hydrochloride, epirubicin, mitomycin, fluorouracil, cisplatin, oxaliplatin, capecitabine, gemcitabine, irinotecan, topotecan, sorafenib, apatinib, lenvatinib, regorafenib, cabozantinib, ramucirumab, nivolumab, and pembrolizumab.

As described above, the drug-loading material provided by the present disclosure can be understood as the polymer material after drug loading, and in addition to containing the crosslinked polymer and a plurality of polyions, the polymer material can also include a bridging agent. Therefore, in some exemplary embodiments, the polymer material included in the drug-loading material further includes a bridging agent, which is used to connect the polyanions to the crosslinked polymer. The role of the bridging agent in the drug-loading material is the same as its role in the polymer material, that is, the bridging agent can serve as an intermediate substance connecting the polyion molecular chains and the crosslinked polymer molecular chains.

In some exemplary embodiments, the bridging agent includes at least two functional groups, and the bridging agent connects the polyelectrolyte to the crosslinked polymer through the at least two functional groups, each of the at least two functional groups includes any one of an amino group, carboxyl group, aldehyde group, mercapto group, carbon-carbon double bond, carbon-carbon triple bond, acryloyl group, isobutyryl group, azido group, epoxy group, vinyl sulfone group, succinimide group, biotin group, dibenzocyclooctyne group, di(p-nitrophenyl) carbonate group, or norbornene group.

In the present disclosure, the chemical properties of the functional groups on the bridging agent differ, and the relative positions of the functional groups on the bridging agent can also vary. Taking a bridging agent containing two functional groups (referred to as a first functional group and a second functional group) as an example, the relative positions of the first functional group and the second functional group on the bridging agent can take various forms. When the first functional group and the second functional group on the bridging agent are relatively far apart (which can also be understood as the first functional group and the second functional group being located at opposite ends of the bridging agent molecular chain), during the process in which the polyion is grafted onto the molecular chain of the crosslinked polymer via the bridging agent, if the molecular chain length of the bridging agent is large, under the spacing effect of the bridging agent, the polyion molecular chain can extend away from the main body of the three-dimensional network structure of the crosslinked polymer and stretch outward, thereby reducing steric hindrance between the polyion and the crosslinked polymer. At this time, in the case where the polyion is a polyanion, the polyanion can expose more negatively charged groups, thereby adsorbing more positively charged drug particles through electrostatic interaction. Correspondingly, the greater the number of drug particles included on the drug-loading material.

In some exemplary embodiments, the bridging agent included in the drug-loading material can be selected from polyethylene glycol derivatives (NH₂-PEG-Acrylate), N-hydroxysuccinimide (NHS), 1-ethyl-(3-dimethylaminopropyl) carbodiimide (EDC), or acrylamide. It should be understood that the bridging agent included in the drug-loading material can also be selected from other substances, as long as it can serve as an intermediate to connect the crosslinked polymer and the polyelectrolyte.

In a fifth aspect, the present disclosure provides a method for preparing a drug-loading material, including: mixing drug particles with a polymer material, the drug particles are adsorbed onto the polymer material under the action of electrostatic forces to obtain the drug-loading material.

In the present disclosure, the method used in the preparation of the polymer material (or embolic agent) is the same as the aforementioned method for preparing the polymer material and will not be repeated herein. After the polymer material (or embolic agent) is prepared, the drug particles can be mixed with the polymer material (or embolic agent). Specifically, the drug particles can be dissolved before being mixed with the polymer material (or embolic agent), and the drug particles are adsorbed onto the polymer material (or embolic agent) under the action of electrostatic forces to obtain the drug-loading material.

In a sixth aspect, the present disclosure further provides a drug-loaded embolic agent, the drug-loaded embolic agent is made of the drug-loading material.

The drug-loading material provided by the present disclosure can be used as a drug-loaded embolic agent. When the drug-loading material provided by the present disclosure is used as a drug-loaded embolic agent, the forming particle size of the drug-loading material can be controlled during its preparation to suit the size of the site where the drug-loaded embolic agent is to be applied.

The following describes the content of the present disclosure in combination with specific examples.

### Example 1

This example uses a combination of a photochemical crosslinking method and an ordinary chemical crosslinking method to prepare a polymer material (embolic agent), including the following steps:

(1) Weigh 50-500 mg of gelatin methacrylate (GelMA) and 0-200 mg of bridging agent-modified PAA (i.e. PAA-PEGMA prepared by modifying PAA with NH₂-PEG-MA at 1:1 molar ratio) into a centrifuge tube, add 1-5 ml of 1X PBS buffer containing 0.3 wt% photoinitiator of lithium phenyl (2,4,6-trimethylbenzoyl) phosphinate (LAP), and dissolve in a 40-65°C water bath to obtain a 5-25 wt% GelMA solution, serving as the aqueous-phase solution;

(2) Weigh 50-500 g of soybean oil into a beaker, add 0-5 g of Span 80, and stir at 100-100 rpm at 40-65°C for half an hour, then centrifuge and take the upper layer of oil as the oil-phase solution;

(3) Add the aqueous-phase solution and the oil-phase solution simultaneously into the beaker, stir at 200-5000 rpm for 10-100 minutes, and irradiate with ultraviolet light simultaneously for curing crosslinking;

(4) According to the above experimental setup, mechanically stir the two-phase mixed solution for 10-100 minutes, turn off the ultraviolet lamp, transfer the resulting solid-liquid mixture to a centrifuge tube, centrifuge to separate and remove the upper layer of oil, add acetone solution, shake to wash, centrifuge to separate the precipitate, after washing add PBS buffer, shake to wash, centrifuge to separate the precipitate, and after washing obtain the polymer material (embolic agent).

FIG. 4 shows a microscopic image of the polymer material (embolic agent) prepared in Example 1, with a scale bar of 200 µm. As shown in FIG. 4, the polymer material (embolic agent) is spherical, and the particle size distribution range is relatively wide.

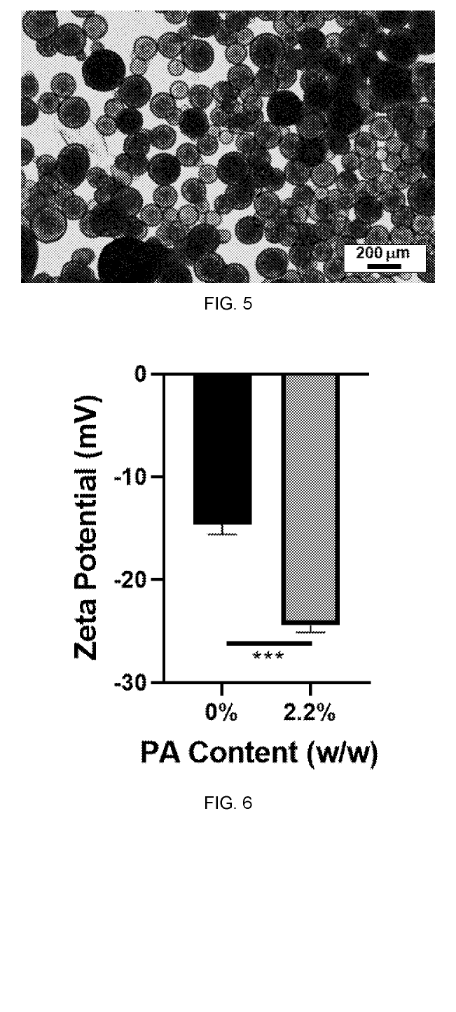

FIG. 5 shows a microscopic image of the polymer material (embolic agent) prepared in Example 1, with a scale bar of 200 µm. As shown in FIG. 5, the polymer material (embolic agent) is spherical, and the particle size distribution range is relatively wide.

Under otherwise identical conditions, the stirring speed during the preparation of the polymer material in FIG. 4 is higher than the corresponding stirring speed during the preparation of the polymer material in FIG. 5. The particle size of the polymer material in FIG. 5 is slightly larger than that of the polymer material in FIG. 4. Therefore, in the present disclosure, the particle size of the prepared polymer material can be controlled by adjusting the stirring speed. For the prepared polymer material, the particle size can later be selected using sieves of different sizes.

After ionization, the polyelectrolyte produces the corresponding polyanion (Polyanion, abbreviated as PA). In the present disclosure, the mass fraction of PA can represent the mass fraction of the polyelectrolyte in the polymer material.

FIG. 6 shows the electronegativity of the polymer material (embolic agent) at different polyacrylic acid contents. As shown in FIG. 6, compared with the polymer material (embolic agent) without polyacrylic acid, the electronegativity of the polymer material (embolic agent) containing polyacrylic acid is significantly increased.

### Example 2

This example uses a microfluidic method combined with photochemical crosslinking to prepare a polymer material (embolic agent), including the following steps:

(1) Weigh 50-500 mg of GelMA and 0-200 mg of PAA-PEGMA into a centrifuge tube, add 1-5 ml of 1X PBS buffer containing 0.3 wt% LAP, and dissolve in a 40-65°C water bath to obtain a 5-25 wt% GelMA solution, serving as the aqueous-phase solution;

(2) Measure 50-500 ml of soybean oil as the oil-phase solution;

(3) Weigh 50-500 g of soybean oil into a beaker, add 0-5 g of Span 80, stir at 100-100 rpm at 40-65°C for half an hour, centrifuge and take the upper layer of oil as the oil-phase solution, serving as the collection solution;

(4) Draw the oil-phase solution and the aqueous-phase solution separately into two 1-20 mL syringes, with the syringe needles connected to silicone tubes (inner diameter 0.5 mm, outer diameter 2 mm), the silicone tube connected to the oil-phase solution is 1-100 cm long, and the silicone tube connected to the aqueous-phase solution is 1-100 cm long; the ends of both silicone tubes are fitted with capillary steel tubes with an outer diameter of 1-100 mm, and the capillary steel tubes are inserted into the injection ports of the “T”-shaped structure of the microfluidic chip.

(5) Place a beaker containing hot water on a heating stirrer, set the temperature of the heating stirrer to 40-100°C, mount the two syringes onto two microsyringe pumps, and set the flow rate of the oil-phase solution to 1-1000 µL/min and the flow rate of the aqueous-phase solution to 1-1000 µL/min;

(6) When the discharged material appears elongated and stable, irradiate the droplets in the channels of the microfluidic chip with ultraviolet light to cure the droplets, immerse the end of the microfluidic chip in the collection solution (the collection solution just covering the outlet of the gelatin embolic agent), stir the collection solution at 80°C and 400 rpm, remove the UV lamp after curing, and wash the microspheres prepared by the emulsion method;

(7) According to the above experimental setup, inject the two-phase solutions for 1 hour, remove the microfluidic chip, transfer the collection solution to a centrifuge tube, centrifuge to separate and remove the upper layer of oil, add acetone solution, shake to wash, centrifuge to separate the precipitate, add PBS buffer, shake to wash, centrifuge to separate the precipitate, and after washing obtain the polymer material (embolic agent).

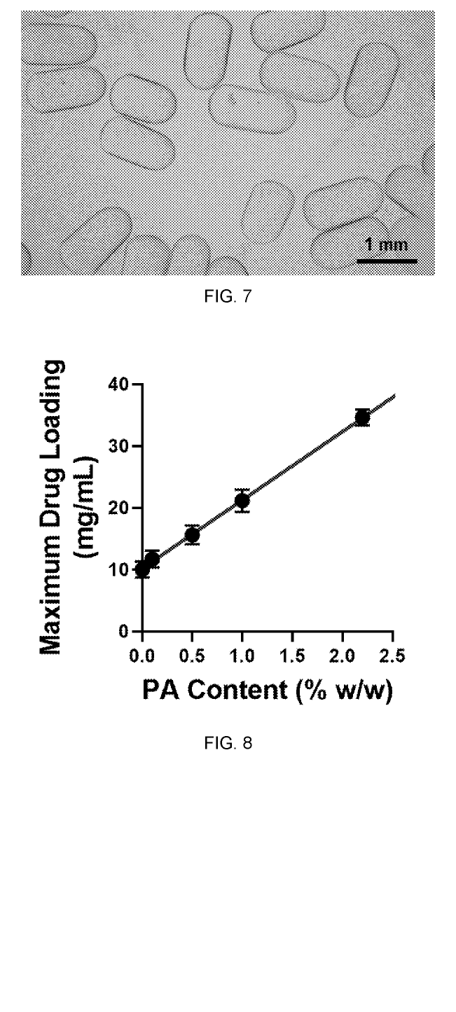

FIG. 7 shows a microscopic image of the polymer material (embolic agent) prepared in Example 2, with a scale bar of 1 mm. As shown in FIG. 7, the polymer material (embolic agent) is elongated, with a length of approximately 400-1000 µm and a width of approximately 300-500 µm.

### Example 3

This example uses a combination of photochemical crosslinking and ordinary chemical crosslinking to prepare a polymer material (embolic agent), including the following steps:

(1) Weigh 50-500 mg of methacrylate-modified starch and 0-200 mg of methacrylated bridging agent-modified carboxyl-based polyelectrolyte (polyaspartic acid) into a centrifuge tube, add 1-5 ml of 1X PBS buffer containing 1 wt% 2-hydroxy-4’-(2-hydroxyethoxy)-2-methylpropiophenone (Irgacure 2959), and dissolve in a 40-65°C water bath to obtain a 5-25 wt% modified starch solution, serving as the aqueous-phase solution;

(2) Weigh 50-500 g of soybean oil into a beaker, add 0-5 g of Span 80, stir at 100-100 rpm at 40-65°C for half an hour, centrifuge, and take the upper layer of oil as the oil-phase solution;

(3) Add the aqueous-phase solution and the oil-phase solution simultaneously into the beaker, stir at 200-5000 rpm for 10-100 minutes, and simultaneously irradiate with ultraviolet light for curing crosslinking;

(4) According to the above experimental setup, mechanically stir the two-phase mixed solution for 10-100 minutes, turn off the ultraviolet lamp, transfer the resulting solid-liquid mixture to a centrifuge tube, centrifuge to separate and remove the upper layer of oil, add acetone solution, shake to wash, centrifuge to separate the precipitate, after washing add PBS buffer, shake to wash, centrifuge to separate the precipitate, and after washing obtain the polymer material (embolic agent).

### Example 4

This example uses a combination of photochemical crosslinking and ordinary chemical crosslinking to prepare a polymer material (embolic agent), including the following steps:

(1) Weigh 50-500 mg of methacrylate modified chondroitin sulfate and 0-200 mg of methacrylated bridging agent-modified carboxyl-based polyelectrolyte (polyglutamic acid) into a centrifuge tube, add 1-5 ml of 1X PBS buffer containing 0.3 wt% LAP, and dissolve in a 40-65°C water bath to obtain a 5-25 wt% modified chondroitin sulfate solution, serving as the aqueous-phase solution;

(2) Weigh 50-500 g of soybean oil into a beaker, add 0-5 g of Span 80, stir at 100-100 rpm at 40-65°C for half an hour, centrifuge, and take the upper layer of oil as the oil-phase solution;

(3) Add the aqueous-phase solution and the oil-phase solution simultaneously into the beaker, stir at 200-5000 rpm for 10-100 minutes, and simultaneously irradiate with ultraviolet light for curing crosslinking;

(4) According to the above experimental setup, mechanically stir the two-phase mixed solution for 10-100 minutes, turn off the ultraviolet lamp, transfer the resulting solid-liquid mixture to a centrifuge tube, centrifuge to separate and remove the upper layer of oil, add acetone solution, shake to wash, centrifuge to separate the precipitate, after washing add PBS buffer, shake to wash, centrifuge to separate the precipitate, and after washing obtain the polymer material (embolic agent).

### Example 5

This example uses the polymer material (embolic agent) prepared in Example 1 to prepare a drug-loading material (drug-loaded embolic agent), including the following steps:

Add 50 mg of doxorubicin hydrochloride to 1 ml of ultrapure water, sonicate for 30 minutes to completely dissolve the doxorubicin hydrochloride in the ultrapure water, then add the doxorubicin hydrochloride aqueous solution to 1 ml of the polymer material (embolic agent) prepared in Example 1, shake on a shaker to allow the doxorubicin hydrochloride to adsorb onto the polymer material (embolic agent), thereby obtaining the drug-loading material (drug-loaded embolic agent).

FIG. 8 shows the maximum drug-loading capacity of the polymer material (embolic agent) corresponding to different mass fractions of polyacrylic acid. As shown in FIG. 8, as the mass fraction of polyacrylic acid gradually increases, the maximum drug-loading capacity of the polymer material (embolic agent) gradually increases, and the mass fraction of polyacrylic acid is positively linearly correlated with the maximum drug-loading capacity of the polymer material (embolic agent).

### Example 6

This example mbodiment uses the polymer material (embolic agent) prepared in Example 1 to prepare a drug-loading material (drug-loaded embolic agent), including the following steps:

Add 25 mg of doxorubicin hydrochloride (an appropriate amount) to 1 ml of ultrapure water, sonicate for 30 minutes to completely dissolve the doxorubicin hydrochloride in the ultrapure water, then add the doxorubicin hydrochloride aqueous solution to 1 ml of the polymer material (embolic agent) prepared in Example 1, shake on a shaker to allow the doxorubicin hydrochloride to adsorb onto the polymer material (embolic agent), thereby obtaining the drug-loading material (drug-loaded embolic agent).

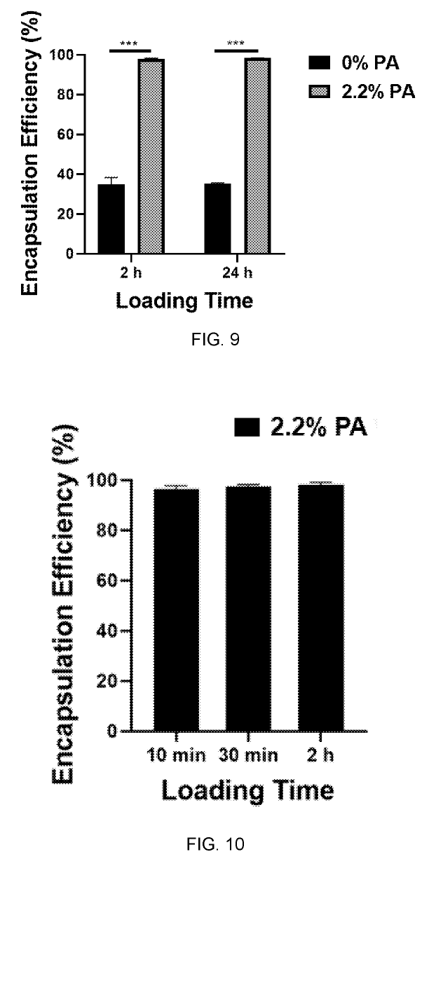

FIG. 9 shows the drug-loading efficiency of the polymer material (embolic agent) corresponding to different mass fractions of polyacrylic acid. As shown in FIG. 9, the polymer material (embolic agent) containing 2.2% polyacrylic acid can adsorb more than 98% of the drug, compared with the polymer material (embolic agent) without polyacrylic acid, which adsorbs less than 40% of the drug, leaving more than 60% of the drug remaining in the aqueous solution.

FIG. 10 shows the change in drug-loading efficiency over time for the polymer material (embolic agent) containing 2.2% (mass fraction) polyacrylic acid. As shown in FIG. 10, the polymer material (embolic agent) containing 2.2% polyacrylic acid can reach a drug-loading efficiency of 97% within 10 minutes, indicating that high-efficiency drug loading can be completed within 10 minutes.

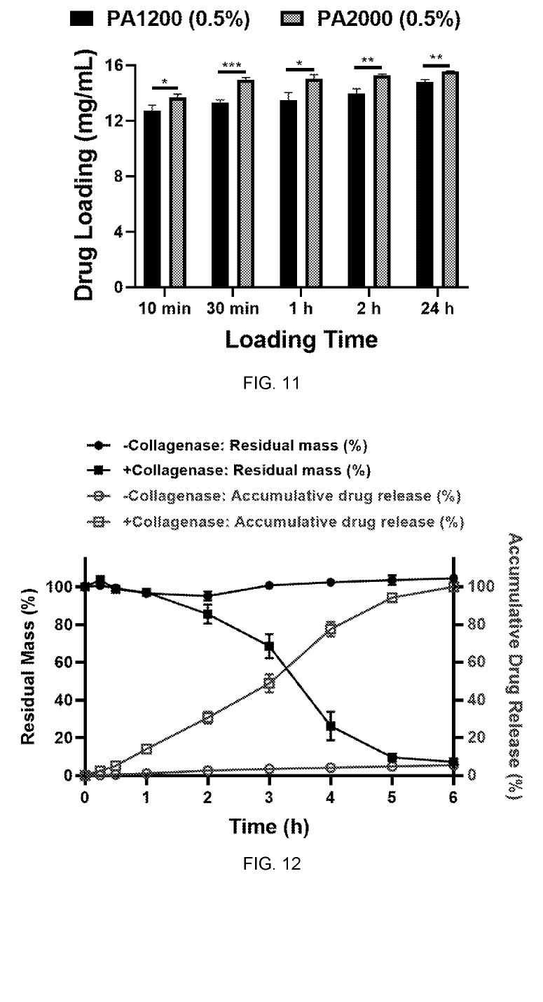

FIG. 11 shows the drug-loading rate of polymer materials (embolic agents) containing the same mass fraction of polyacrylic acid but with different molecular weights. Under the condition of the same mass fraction of polyacrylic acid (i.e., the same number of acidic groups), the number of macromolecular chains corresponding to polyacrylic acid with a molecular weight of 2000 Da is lower than that corresponding to polyacrylic acid with a molecular weight of 1200 Da. Accordingly, one polyacrylic acid molecule with a molecular weight of 2000 Da contains more acidic groups, and the polyanion formed after ionization has a higher density of anionic groups. As shown in FIG. 11, in polymer materials containing the same mass fraction of polyacrylic acid, compared with polymer materials using polyacrylic acid with a molecular weight of 1200 Da, polymer materials using polyacrylic acid with a molecular weight of 2000 Da exhibit a faster drug-loading rate and a higher drug-loading capacity. This indicates that higher-molecular-weight polyanions, having a higher density of anionic groups, can exhibit a stronger counterion condensation effect.

FIG. 12 shows the degradation rate and drug release rate of the same drug-loading material (drug-loaded embolic agent) under different in vitro conditions. As shown in FIG. 12, under enzyme-catalyzed conditions, the drug-loading material (drug-loaded embolic agent) can undergo degradation, and under these conditions, the drug can be released in an essentially linear manner (the drug release rate is roughly proportional to time), achieving complete drug release.

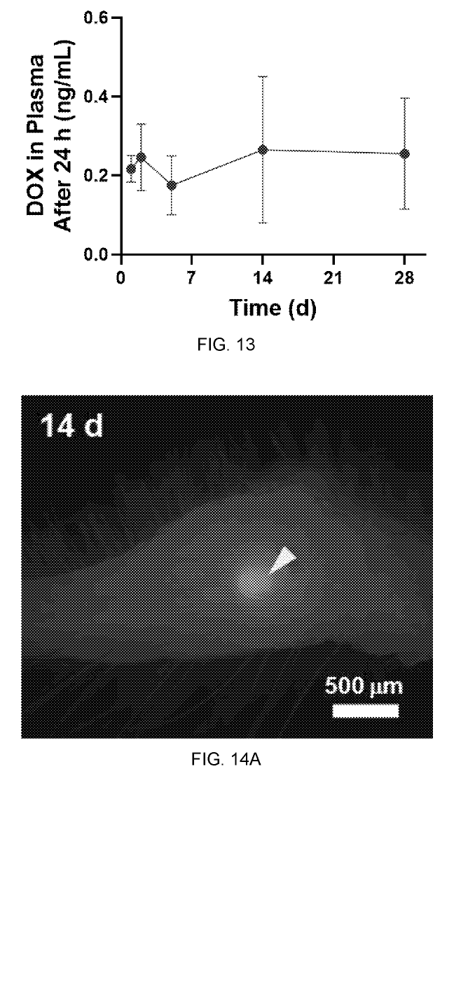

FIG. 13 shows the drug release profile of a drug-loading material (drug-loaded embolic agent) in transarterial chemoembolization in rabbit ear vessels in this example. As shown in FIG. 13, the drug-loading material continuously degrades and releases the drug in vivo, with plasma drug concentrations remaining relatively constant from 24 hours up to 28 days.

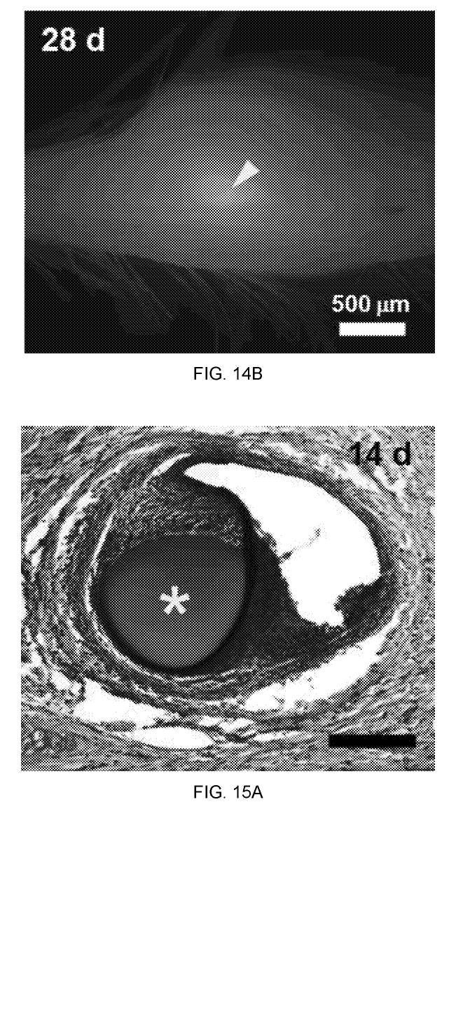

FIG. 14 (including FIGS. 14A and 14B) shows the fluorescence signal changes of a drug-loading material (drug-loaded embolic agent) in transarterial chemoembolization in rabbit ear vessels in this example. The arrows indicate the location of the embolic material (i.e., the embolization site), and the scale bar in FIG. 14 is 500 µm.

FIG. 14A shows the fluorescence signal of the drug-loading material (drug-loaded embolic agent) on day 14 of transarterial chemoembolization in rabbit ear vessels; FIG. 14B shows the fluorescence signal on day 28. As shown in FIGS. 14A and 14B, because the drug-loading material continuously degrades and releases the drug in vivo, the fluorescence signal of the drug in the tissue outside the embolized vessel does not diminish over time. In fact, compared with day 14 (FIG. 14A), the fluorescence signal corresponding to the drug in the local tissue on day 28 (FIG. 14B) is enhanced.

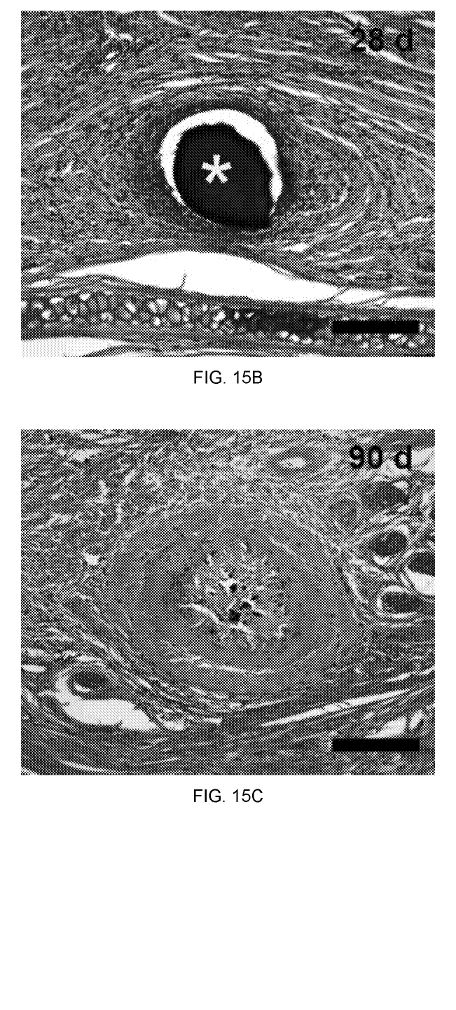

FIG. 15 (including FIGS. 15A-15C) shows H&E-stained tissue sections corresponding to different time points of a drug-loading material (drug-loaded embolic agent) in transarterial chemoembolization in rabbit ear vessels. The “*” indicates the location of the drug-loading material (i.e., the embolization site), and the scale bar in FIG. 15 is 200 µm. FIG. 15A shows the H&E-stained tissue section on day 14; FIG. 15B shows the H&E-stained tissue section on day 28; FIG. 15C shows the H&E-stained tissue section on day 90. As shown in FIGS. 15A-15C, the drug-loading material in the embolized vessel gradually degrades over time and is completely degraded after 90 days. The H&E-stained tissue sections show that compared with days 14 (FIG. 15A) and 28 (FIG. 15B), after complete degradation of the embolic material on day 90 (FIG. 15C), the number of immune cells around the embolization site is greatly reduced, indicating that complete degradation of the drug-loading material helps reduce immune inflammatory responses.

### Example 7

This example uses the polymer material (embolic agent) prepared in Example 2 to prepare a drug-loading material (drug-loaded embolic agent), including the following steps:

Add 25 mg of doxorubicin hydrochloride (an appropriate amount) to 1 ml of ultrapure water, sonicate for 30 minutes to completely dissolve the doxorubicin hydrochloride in the ultrapure water, then add the doxorubicin hydrochloride aqueous solution to 1 ml of the polymer material (embolic agent) prepared in Example 2, shake on a shaker to allow the doxorubicin hydrochloride to adsorb onto the polymer material (embolic agent), thereby obtaining the drug-loading material (drug-loaded embolic agent).

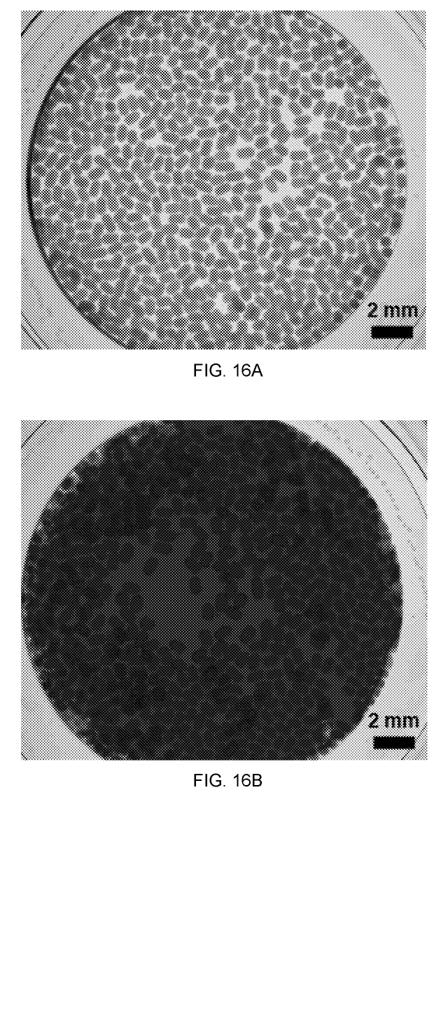

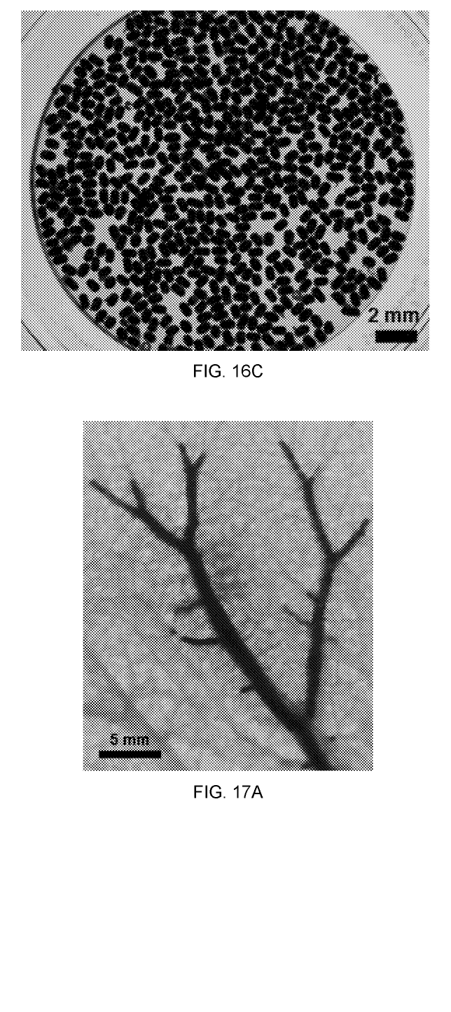

FIG. 16 (including FIGS. 16A-16C) shows the changes of the polymer material (embolic agent) from before drug loading to after drug loading. The scale bar in FIG. 16 is 2 mm. FIG. 16A shows a microscopic image of the polymer material (embolic agent) before drug loading. FIG. 16B shows a microscopic image immediately after mixing doxorubicin hydrochloride with the polymer material (embolic agent). Because the doxorubicin hydrochloride solution itself is colored (orange-red), the mixture initially exhibits the same color as the doxorubicin hydrochloride solution. FIG. 16C shows a microscopic image of the polymer material (embolic agent) after drug loading, resulting in the drug-loading material (drug-loaded embolic agent). At this point, because the doxorubicin hydrochloride has been adsorbed into the polymer material (embolic agent), the originally colorless polymer material (embolic agent) becomes dark red, thereby forming the drug-loading material (drug-loaded embolic agent).

### Example 8

In this example, the drug-loading material (drug-loaded embolic agent) prepared in Example 7 is injected into a decellularized rabbit liver using a 10 cm flat-tip needle to observe its embolization and drug-loading effects.

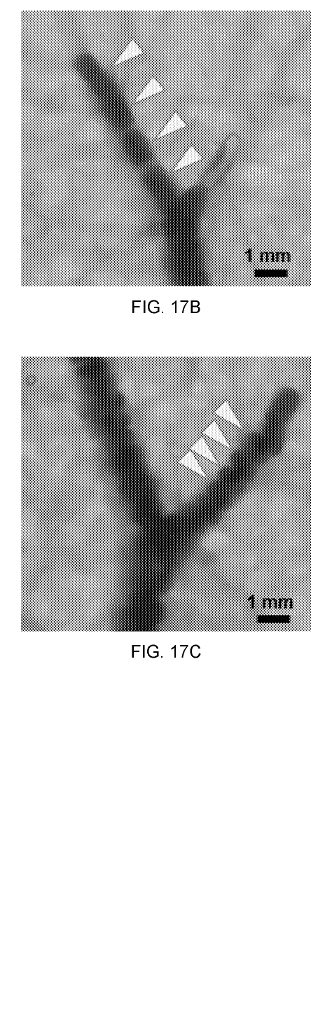

FIG. 17A shows the arrangement of the drug-loading material (drug-loaded embolic agent) in a blood vessel, demonstrating the effect of drug release along the vessel. The scale bar in FIG. 17A is 5 mm. FIG. 17B shows the arrangement of the drug-loading material (drug-loaded embolic agent) in a blood vessel, demonstrating the effect of drug release along the vessel. The scale bar in FIG. 17B is 1 mm. FIG. 17C shows the arrangement of the drug-loading material (drug-loaded embolic agent) in a blood vessel, demonstrating the effect of drug release along the vessel. The scale bar in FIG. 17C is 1 mm.

As shown in FIGS. 17A-17C, the drug-loading material (drug-loaded embolic agent) prepared in Example 7 can align linearly at the distal end of the blood vessel to achieve deeper embolization. The material is densely arranged and stably distributed within the blood vessel, making it unlikely to shift position. At the same time, the drug-loading material (drug-loaded embolic agent) prepared in Example 7 allows the slow release of doxorubicin to be observed in the blood vessel through circulation of physiological saline.

The technical features described in the above embodiments can be combined in any manner. For brevity, not all possible combinations of the technical features in the above embodiments are described; however, as long as these combinations of technical features do not conflict, they are considered to fall within the scope disclosed in the present disclosure.

The above embodiments merely illustrate several examples of the present disclosure, and their descriptions are relatively specific and detailed; however, this should not be understood as a limitation on the scope of the patent. It should be noted that, for a person skilled in the art, various modifications and improvements can be made without departing from the concept of the present disclosure, and these are all within the protection scope. It should be understood that any technical solutions obtained by a person skilled in the art, based on the technical solutions provided by the present disclosure through logical analysis, reasoning, or limited experimentation, also fall within the protection scope of the appended claims. Therefore, the protection scope of the present patent should be defined by the appended claims, and the specification and drawings can be used to interpret the content of the claims.

What is claimed is:

1. A polymeric material, comprising:
a crosslinked polymer; and
a plurality of polyelectrolytes grafted via chemical bonds onto a three-dimensional network structure of the crosslinked polymer, wherein each of the polyelectrolytes comprises a plurality of ionizable groups, and the ionizable groups form a local potential after ionization to induce aggregation of particles having an opposite electric charge.

2. The polymeric material according to claim 1, wherein each of the polyelectrolytes comprises the plurality of ionizable groups, the plurality of ionizable groups forms a plurality of anionic groups after ionization, and the plurality of anionic groups forms a local low potential capable of inducing aggregation of positively charged particles.

3. The polymeric material according to claim 1, wherein each of the polyelectrolytes comprises 3 or more ionizable groups.

4. The polymeric material according to claim 2, wherein the ionizable groups comprise at least one of a carboxyl group, a sulfonic acid group, a sulfinic acid group, a phosphoric acid group, a phosphorous acid group, or a hypophosphorous acid group.

5. The polymeric material according to claim 1, wherein the polyelectrolytes comprise at least one of polyacrylic acid, carboxymethyl cellulose, carboxymethyl chitosan, sodium alginate, G4 dendrimer, polyglutamic acid, or polyaspartic acid.

6. The polymeric material according to claim 1, wherein the crosslinked polymer is a degradable crosslinked polymer.

7. The polymeric material according to claim 6, wherein the degradable crosslinked polymer comprises at least one of crosslink-modified gelatin, crosslink-modified chitosan, crosslink-modified hyaluronic acid, crosslink-modified agarose, crosslink-modified chondroitin sulfate, crosslink-modified starch, or crosslink-modified cellulose.

8. The polymeric material according to claim 1, wherein the crosslinked polymer accounts for 1-30.% by mass, and the plurality of polyelectrolytes accounts for 0.2-30.% by mass.

9. The polymeric material according to claim 1, further comprising:
a bridging agent, between the polyelectrolytes and the crosslinked polymer, to connect the polyelectrolytes with the crosslinked polymer.

10. The polymeric material according to claim 9, wherein the bridging agent comprises at least two functional groups, and the bridging agent connects the polyelectrolytes with the crosslinked polymer through the at least two functional groups, wherein
each of the at least two functional groups comprises any one of an amino group, a carboxyl group, an aldehyde group, a mercapto group, a carbon-carbon double bond, a carbon-carbon triple bond, an acryloyl group, an isobutenoyl group, an azido group, an epoxy group, a vinylsulfonyl group, a succinimide group, a biotin group, a dibenzocyclooctyne group, a di(p-nitrophenyl) carbonate group, or a norbornene group.

11. A method for preparing a polymeric material, comprising:
mixing a crosslinking precursor, a crosslinking agent, and polyelectrolytes; and
performing a crosslinking polymerization reaction under an external condition stimulation to form the polymeric material, wherein polymeric material comprises: a crosslinked polymer, and a plurality of the polyelectrolytes grafted via chemical bonds onto a three-dimensional network structure of the crosslinked polymer, wherein each of the polyelectrolytes comprises a plurality of ionizable groups, so that the ionizable groups form a local potential after ionization to induce aggregation of particles having an opposite electric charge.

12. The preparation method according to claim 11, wherein the mixing of the crosslinking precursor, the crosslinking agent, and the polyelectrolytes comprises:
mixing the crosslinking precursor, the crosslinking agent, the polyelectrolytes, and a bridging agent.

13. The preparation method according to claim 11, wherein in the polymeric material, the bridging agent is between the polyelectrolytes and the crosslinked polymer to connect the polyelectrolytes with the crosslinked polymer.

14. A drug-loading material, comprising:
a polymeric material, comprising: a crosslinked polymer, and a plurality of polyions grafted by chemical bonds onto a three-dimensional network structure of the crosslinked polymer; and
a plurality of drug particles, adsorbed onto the polymeric material through electrostatic interaction, wherein
each of the polyions comprises a plurality of charged groups, and the plurality of charged groups forms a local potential to induce aggregation of the drug particles having opposite electric charges.

15. The drug-loading material according to claim 14, wherein the plurality of polyions comprises: a plurality of polyanions, grafted by chemical bonds onto the three-dimensional network structure of the crosslinked polymer; and
the plurality of drug particles comprises: a plurality of positively charged drug particles, adsorbed onto the polymeric material through electrostatic interaction, wherein
each of the polyanions comprises a plurality of negatively charged groups, and the plurality of negatively charged groups forms a local low potential to induce aggregation of the plurality of positively charged drug particles.

16. The drug-loading material according to claim 15, wherein the drug particles are locally aggregated and adsorbed onto the plurality of polyanions of the polymeric material through electrostatic interaction.

17. The drug-loading material according to claim 16, wherein the drug particles are further adsorbed, through electrostatic interaction, onto the negatively charged groups carried by the crosslinked polymer.

18. The drug-loading material according to claim 14, wherein the drug particles comprise at least one of doxorubicin hydrochloride, epirubicin, mitomycin, fluorouracil, cisplatin, oxaliplatin, capecitabine, gemcitabine, irinotecan, topotecan, sorafenib, apatinib, lenvatinib, regorafenib, cabozantinib, ramucirumab, nivolumab, and pembrolizumab.

19. The drug-loading material according to claim 14, wherein the polymeric material further comprises a bridging agent to connect the polyanions with the crosslinked polymer.

20. The drug-loading material according to claim 19, wherein the bridging agent comprises at least two functional groups, and the bridging agent connects the polyelectrolytes with the crosslinked polymer through the at least two functional groups, wherein
each of the at least two functional groups comprises any one of an amino group, a carboxyl group, an aldehyde group, a mercapto group, a carbon-carbon double bond, a carbon-carbon triple bond, an acryloyl group, an isobutenoyl group, an azido group, an epoxy group, a vinyl sulfone group, a succinimide group, a biotin group, a dibenzocyclooctyne group, a di(p-nitrophenyl)carbonate group, or a norbornene group.
